(19) United States
(12) Patent Application Publication
(10) Pub. No.: US 2025/0213730 A1
(43) Pub. Date: Jul. 3, 2025

(54) GENE THERAPY FOR DISEASES WITH CNS MANIFESTATIONS

(71) Applicant: TAKEDA PHARMACEUTICAL COMPANY LIMITED, Osaka (JP)

(72) Inventors: Rizwana ISLAM, Cambridge, MA (US); Mugdha DESHPANDE, Cambridge, MA (US); Sarah Melissa JACOBO, Cambridge, MA (US); Wanida RUANGSIRILUK, Cambridge, MA (US)

(21) Appl. No.: 18/853,011

(22) PCT Filed: Mar. 31, 2023
(86) PCT No.: PCT/IB2023/053220

§ 371 Date: Sep. 30, 2024

Related U.S. Application Data
(60) Provisional application No. 63/478,342, filed on Jan. 3, 2023.
(60) Provisional application No. 63/350,822, filed on Jun. 9, 2022.
(60) Provisional application No. 63/326,780, filed on Apr. 1, 2022.

(51) Int. Cl. A61K 48/00; A61P 3/02; C07K 16/28; C12N 9/14

(52) U.S. Cl. CPC A61K 48/0058; A61P 3/02; C07K 16/2881; C12N 9/14; C12Y 310/01001; C07K 2317/569; C07K 2319/01

(57) ABSTRACT

The present disclosure relates generally to viral vectors comprising an engineered transgene capable of crossing the blood brain barrier and uses thereof in the treatment of diseases presenting with central nervous system manifestations, such as, but not limited to Hunter syndrome, Gaucher disease, and Sanfilippo syndrome.

### CROSS REFERENCE TO RELATED APPLICATIONS

This PCT application claims the priority benefit of U.S. Provisional Application Nos. 63/326,780, filed Apr. 1, 2022; 63/350,822, filed Jun. 9, 2022; and 63/478,342, filed Jan. 3, 2023, each of which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 3817_141PC03_SequenceListing_ST26; Size: 41,454 bytes; and Date of Creation: Mar. 30, 2023), filed with the application, is incorporated herein by reference in its entirety.

### FIELD

The present disclosure relates generally to viral vectors comprising an engineered transgene capable of crossing the blood brain barrier and uses thereof in the treatment of diseases presenting with central nervous system manifestations.

### BACKGROUND

Diseases that affect the central nervous system (CNS) are some of the most challenging diseases to treat. The body's own protection, including the blood brain barrier (BBB), makes it very difficult if not impossible for systemically delivered large drugs and biologics to have an effect within the CNS. The use of protein-based therapies in particular has been limited by minimal brain exposure (see, e.g., Arguello and Mahon et al., J. Exp. Med. 219(3):e20211057 (2022); Kumar et al., Bioconjug. Chem.; 29(12):3937-3966 (2018); Stanimirovic et al., BioDrugs. 32(6):547-559 (2018)). Similarly, most polar small molecules, such as antibodies, and nearly all macromolecules are effectively restricted from infiltrating the brain in therapeutically relevant concentrations by physical and biochemical barriers, most notably the BBB (Arguello and Mahon et al., J. Exp. Med. 219(3):e20211057 (2022); Abbott et al., Acta. Neuropathol. (2018); Banks, Nat. Rev. Drug Discov. 15(4):275-92 (2016)).

This hurdle to effective treatment is particularly relevant for lysosomal storage disorders (LSDs). LSDs represent a family of more than 50 monogenic diseases, many of which are characterized by a defect in a single lysosomal enzyme. Their combined prevalence is estimated to be 1 in 8,000 births (see Arguello and Mahon et al., J. Exp. Med. 219(3):e20211057 (2022); Schultz et al., Trends Neurosci. 34(8):401-10 (2011)), constituting a significant underserved patient population. Disease-associated variants lead to a reduction or loss of enzymatic activity, resulting in substrate accumulation and broad lysosomal dysfunction (Platt et al., J. Cell Biol. 199(5):723-734 (2012)), which can trigger pathogenic cascades affecting multiple tissues throughout the body, including the CNS (Bellettato and Scarpa, J. Inherit. Metab. Dis. 33(4):347-62 (2010)). In fact, given the ubiquity of lysosomes in all cells of the body, in addition to the syndromic manifestations in peripheral organs, most LSDs involve progressive CNS impairment. However, treatment of CNS manifestations in LSDs remains a considerable challenge.

The standard of care for many LSDs is a systemically administered recombinant enzyme replacement therapy (ERT); however, these first generation enzymes do not readily cross the BBB and typically have been ineffective in treating the CNS manifestations of disease (Scarpa et al., Best Pract. Res. Clin. Endocrinol. Metab. 29(2):159-71 (2015)). Despite promising reductions in substrate levels outside the CNS, these therapies are often less effective in reducing substrate levels in the brain (See, e.g., Sonoda et al., Mol. Ther. 26:1366-74 (2018); Morimoto et al., Mol. Ther. 29:1853-61 (2021)). In addition to the paucity of treatments that effectively address the neurological manifestations of these diseases, direct CNS delivery approaches are invasive, susceptible to device issues, and have the potential for suboptimal brain distribution (Ullman et al. Sci. Transl. Med. 12(545):1163 (2020)).

For example, Mucopolysaccharidosis II (MPS II), also known as Hunter syndrome, a lysosomal storage disease caused by mutations in iduronate-2-sulfatase (IDS or I2S), is characterized by a wide variety of somatic and neurologic symptoms. Hunter syndrome is a debilitating disease that affects multiple organ systems, including organomegaly (particularly of the liver and spleen), progressive joint and skeletal involvement, and, in the neuronopathic form of MPS II, severe cognitive deficits. The currently approved intravenous ERT with recombinant IDS has been ineffective at treating the CNS manifestations due to its inability to cross the BBB (Noh and Lee, J. Clin. Pharm. Ther. 39(3):215-24 (2014)).

The mucopolysaccharidoses (MPS) are a group of inherited lysosomal storage disorders, and in addition to Hunter syndrome, include, for example, Hurler syndrome (MPS I), Sanfilippo syndrome (MPS III), and Sly syndrome (MPS VII). Hurler syndrome is the most severe form of mucopolysaccharidosis and is characterized by a deficiency of the enzyme alpha-L-iduronidase, which results in accumulation of dermatan and heparan sulfates. Sanfilippo syndrome has four subtypes (A, B, C, and D), which are distinguished by four different enzyme deficiencies, and Sly syndrome is characterized by a deficiency of beta-glucoronidase, resulting in the accumulation of the glycosaminoglycans, dermatan sulfate, heparan sulfate, and chondroitin sulfate. Other LSDs include, for example, Gaucher disease, Metachromatic leukodystrophy, Krabbe disorder, and GM1 gangliosidosis. Gaucher disease, for example, is characterized by a deficiency of glucocerebrosidase, which results in accumulation of certain lipids, specifically glucocerebroside, throughout the body particularly in the bone marrow, spleen, and liver. The disease may also affect the brain, lungs, eyes, and bones, thus symptoms vary.

An emerging strategy to improve brain delivery of protein therapeutics has been to exploit receptor-mediated transcytosis (RMT) (Ullman et al. Sci. Transl. Med. 12(545): 1163 (2020)). RMT is an endogenous process wherein essential biomolecules that cannot passively diffuse into the brain from the bloodstream (e.g., insulin and transferrin-bound iron) are actively transported across the BBB through specific interactions with brain endothelial cell receptors (Johnsen et al., Prog. Neurobiol. 2019 181:101665 (2019)). The transferrin receptor (TfR) is an RMT target at the BBB, due, in part, to its enriched expression on brain endothelial cells (Jefferies et al., Nature. 312:162-163 (1984)). However, a number of imaging and biodistribution studies in mouse models have suggested that antibodies targeting TfR may be only minimally released into the brain parenchyma (Paris-Robidas et al., Mol Pharmacol. 80(1):32-9 (2011); Paterson and Webster, Drug Discov. 20:49-52 (2016)).

Accordingly, for patients with CNS manifestations of diseases caused by insufficiency of a certain enzyme or protein, there is a need in the art for therapeutics that result in widespread CNS parenchymal exposure of the specific enzyme or protein. In LSDs specifically, there is a need in the art for therapeutics that increase CNS penetration to ultimately reduce substrate accumulation.

### SUMMARY OF THE DISCLOSURE

Some aspects of the present disclosure are directed to a gene therapy vector comprising: (a) a 5′ inverted terminal repeat (ITR) (b) a promoter, (c) a transgene comprising (i) a nucleotide sequence encoding a biologically active polypeptide and (ii) a nucleotide sequence encoding a TAG, and (d) a 3′ ITR.

In some aspects, the biologically active polypeptide comprises a therapeutic enzyme. In some aspects, the biologically active polypeptide reduces the levels of a substrate in the central nervous system. In some aspects, the biologically active polypeptide comprises an idursulfase activity, comprises a glucocerebrosidase activity, comprises a Sulfoglucosamine Sulfohydrolase activity, or comprises a sulfamidase activity. In some aspects, the biologically active polypeptide is an idursulfase (IDS), a glucocerebrosidase (GCB), a N-Sulfoglucosamine Sulfohydrolase, or a sulfamidase (SGSH).

In some aspects, the TAG increases the translocation of the biologically active polypeptide across the blood brain barrier. In some aspects, the TAG comprises an antigen-binding molecule. In some aspects, the TAG comprises an scFv, a VHH, a vNAR, a diabody, a nanobody, a camelid antibody, or a combination thereof. In some aspects, the TAG comprises a VHH. In some aspects, the TAG comprises an antigen-binding molecule that specifically binds transferrin receptor 1 (TfR1). In some aspects, the TAG comprises a VHH that specifically binds TfR1.

In some aspects, the TAG comprises a variable heavy (VH) domain comprising a VH complementarity determining region (CDR) 1, a VH-CDR2, and a VH-CDR3. In some aspects, the VH-CDR1 is encoded by the nucleic acid sequence set forth in SEQ ID NO: 11. In some aspects, the VH-CDR2 is encoded by the nucleic acid sequence set forth in SEQ ID NO: 12. In some aspects, the VH-CDR3 is encoded by the nucleic acid sequence set forth in SEQ ID NO: 13. In some aspects, the nucleotide sequence encoding the TAG comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% identity to SEQ ID NO: 14, 15, 16, 17, 18, 19, 20, or 21. In some aspects, the nucleotide sequence encoding the TAG comprises SEQ ID NO: 14, 15, 16, 17, 18, 19, 20, or 21. In some aspects, the nucleotide sequence encoding the TAG comprises SEQ ID NO: 14. In some aspects, the nucleotide sequence encoding the TAG comprises SEQ ID NO: 15.

In some aspects, the nucleotide sequence encoding the biologically active polypeptide is 3′ of the nucleotide sequence encoding the TAG. In some aspects, the nucleotide sequence encoding the biologically active polypeptide is 5′ of the nucleotide sequence encoding the TAG.

In some aspects, the (i) nucleotide sequence encoding the biologically active polypeptide is linked to the (ii) nucleotide sequence encoding the TAG further by (iii) a nucleotide sequence encoding a peptide linker. In some aspects, the linker is a flexible linker, a cleavable linker, a processable linker, or any combination thereof.

In some aspects, the promoter is a ubiquitous promoter. In some aspects, the ubiquitous promoter comprises a chicken β actin (CBA) promoter, an EF-1α promoter, a PGK promoter, a UBC promoter, an LSE beta-glucuronidase (GUSB) promoter, or a ubiquitous chromatin opening element (UCOE) promoter. In some aspects, the ubiquitous promoter comprises a cyto-megalo-virus (CMV) enhancer, a chicken β actin promoter (CBA), and a rabbit beta globin intron.

In some aspects, the promoter is a tissue specific promoter. In some aspects, the promoter is a liver specific promoter. In some aspects, the promoter comprises an hTTR, PGK, chicken β actin (CBA) promoter, CAG promoter, EF-1α promoter, UBC promoter, LSE beta-glucuronidase (GUSB) promoter, or ubiquitous chromatin opening element (UCOE) promoter, or any combination thereof.

In some aspects, the gene therapy vector is a recombinant AAV (rAAV). In some aspects, the rAAV comprises an AAV capsid. In some aspects, the AAV capsid is a wide-tropism AAV capsid. In some aspects, the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, AAV9 capsid, and a variant thereof. In some aspects, the AAV capsid is AAV9.

In some aspects, the gene therapy vector further comprises a polyA sequence, which is located 3′ of the transgene. In some aspects, the poly A is bovine growth hormone (BGH) polyA or a synthetic polyA. In some aspects, the polyA is a synthetic polyA that is designed in silico. In some aspects, the gene therapy vector further comprises a posttranscriptional regulatory element. In some aspects, the posttranscriptional regulatory element is located 3′ of the transgene. In some aspects, the posttranscriptional regulatory element is located 5′ of the polyA sequence. In some aspects, the posttranscriptional regulatory element comprises a woodchuck hepatitis virus posttranscriptional regulatory element (WPRE). In some aspects, the WPRE sequence is modified. In some aspects, the WPRE sequence is WPRE mut6delATG. In some aspects, the promoter comprises a shortened EF-1α promoter and one or more introns. In some aspects, the one or more introns are from CBA and/or rabbit β-globin genes.

In some aspects, the transgene is codon optimized.

Some aspects of the present disclosure are directed to a method of treating a disease or condition in a subject in need thereof, comprising administering to the subject the gene therapy vector encompassed by the present disclosure. In some aspects, the disease or condition comprises a neuronal disease. In some aspects, the disease or condition comprises a mucopolyscahharidoses. In some aspects, the disease or condition comprises Hurler syndrome (MPS I), Hunter syndrome (MPS II), Sanfilippo syndrome (MPS III), Sly syndrome (MPS VII), Gaucher disease, Metachromatic leukodystrophy, Krabbe disorder, and GM1 gangliosidosis.

Some aspects of the present disclosure are directed to a method of delivering a biologically active polypeptide across the blood brain barrier in a subject in need thereof, comprising administering to the subject the gene therapy vector encompassed by the present disclosure, wherein such administration results in reducing the level of a substrate in the central nervous system of the subject. Some aspects of the present disclosure are directed to a method of substrate reduction in the central nervous system of a subject, comprising administering to the subject a gene therapy vector disclosed herein.

Some aspects of the present disclosure are directed to a method of treating a mucopolyscahharidoses in a subject in need thereof, comprising administering to the subject a gene therapy vector disclosed herein. Some aspects of the present disclosure are directed to a method of treating Hurler syndrome (MPS I) in a subject in need thereof, comprising administering to the subject a gene therapy vector disclosed herein. Some aspects of the present disclosure are directed to a method of treating Hunter syndrome (MPS II) in a subject in need thereof, comprising administering to the subject a gene therapy vector disclosed herein. Some aspects of the present disclosure are directed to a method of treating Sanfilippo syndrome (MPS III) in a subject in need thereof, comprising administering to the subject a gene therapy vector disclosed herein. Some aspects of the present disclosure are directed to a method of treating Sly syndrome (MPS VII) in a subject in need thereof, comprising administering to the subject a gene therapy vector disclosed herein. Some aspects of the present disclosure are directed to a method of treating Gaucher disease in a subject in need thereof, comprising administering to the subject a gene therapy vector disclosed herein. Some aspects of the present disclosure are directed to a method of treating Metachromatic leukodystrophy in a subject in need thereof, comprising administering to the subject a gene therapy vector disclosed herein. Some aspects of the present disclosure are directed to a method of treating Krabbe disorder in a subject in need thereof, comprising administering to the subject a gene therapy vector disclosed herein. Some aspects of the present disclosure are directed to a method of treating GM1 gangliosidosis in a subject in need thereof, comprising administering to the subject a gene therapy vector disclosed herein.

In some aspects, a recombinant polypeptide is expressed from the transgene outside the central nervous system, and the TAG facilitates translocation of the recombinant polypeptide across the blood brain barrier into the central nervous system.

In some aspects, the recombinant polypeptide reduces the level of a substrate in the central nervous system of the subject. In some aspects, the recombinant polypeptide reduces the level of a substrate systemically.

Some aspects of the present disclosure are directed to a method of treating Hunter syndrome in a subject in need thereof, comprising administering to the subject a gene therapy vector disclosed herein, wherein the biologically active polypeptide comprises IDS.

Some aspects of the present disclosure are directed to a method of treating Gaucher disease in a subject in need thereof, comprising administering to the subject a gene therapy vector disclosed herein, wherein the biologically active polypeptide comprises glucocerebrosidase (GCB).

Some aspects of the present disclosure are directed to a method of treating Sanfilippo syndrome in a subject in need thereof, comprising administering to the subject a gene therapy vector disclosed herein, wherein the biologically active polypeptide comprises N-Sulfoglucosamine Sulfohydrolase.

### BRIEF DESCRIPTION OF THE DRAWINGS/FIGURES

FIGS. 1A-1C are graphical representations of brain I2S activity (nmol/hr/mg of total protein, FIG. 1A), brain heparan sulfate levels (ng/mg of total prot., FIG. 1B), and CSF heparan sulfate levels (ng/mL CSF, FIG. 1C) for 8-12 weeks old male IdsKO mice following single intravenous administration of liver directed gene therapy constructs [rAAV9-GTH077 (I2S), rAAV9-GTH071 (I2S-VHH) or rAAV9-GTH074 (VHH-I2S)] at 2.5×10¹² vg/kg and 6.25×10¹² vg/kg. Data for control rAAV9-MY011 (null) treated and wild-type (WT:WT) littermate vehicle treated groups are also shown.

FIGS. 2A-2H are representative micrographs of a thalamic neuron immunostained for I2S (FIGS. 2A-2D) and LAMP1 (FIGS. 2E-2H) from mice injected with liver driven GT constructs: rAAV9-GTH077 (I2S; FIGS. 2A and 2E), rAAV9-GTH074 (VHH-I2S; FIGS. 2B and 2F), or rAAV9-GTH071 (I2S-VHH; FIGS. 2C and 2G); and a negative control (FIGS. 2D and 2H).

FIGS. 3A-3L are representative micrographs of LAMP1 immunostaining in the cortex (FIGS. 3A-3D), hippocampus (FIGS. 3E-3H), and thalamus (FIGS. 3I-3L) of IdsKO mice following intravenous administration of rAAV9-GTH077 (I2S; FIGS. 3A, 3E, and 3I) or rAAV9-GTH074 (VHH-I2S; FIGS. 3B, 3F, and 3J) compared to LAMP1 immunostaining in IdsKO control (FIGS. 3C, 3G, and 3K) and WT control (FIGS. 3D, 3H, and 3L) mice in the same brain regions.

FIGS. 4A-4F are graphical representations of hI2S activity (nmol/hr/mg of total protein) in terminal serum (FIG. 4A), liver (FIG. 4B), lung (FIG. 4C), bone marrow (FIG. 4D), heart (FIG. 4E), and kidney (FIG. 4F) detected after administering rAAV9-GTH077 (I2S), rAAV9-GTH071 (I2S-VHH) or rAAV9-GTH074 (VHH-I2S) at 2.5×10¹² vg/kg and 6.25×10¹² vg/kg to IdsKO mice.

FIGS. 5A-5C are graphical representations of brain I2S activity (nmol/hr/mg of total protein; FIG. 5A), brain heparan sulfate levels (ng/mg of total protein; FIG. 5B), and CSF heparan sulfate levels (ng/mL CSF; FIG. 5C) for animals administered rAAV9 vectors expressing I2S driven by a ubiquitous promoter [rAAV9-GTH075 (I2S), rAAV9-GTH069 (I2S-VHH), and rAAV9-GTH072 (VHH-I2S)] at a 2.5×10¹² vg/kg dose. Data for control rAAV9-MY011 (null) treated and wild-type (WT:WT) littermate vehicle treated groups are also shown.

FIGS. 6A-6F are graphical representations of hI2S activity in terminal serum (FIG. 6A), liver (FIG. 6B), lung (FIG. 6C), bone marrow (FIG. 6D), heart (FIG. 6E), and kidney (FIG. 6F) after administering to IdsKO mice rAAV9-GTH075 (I2S), rAAV9-GTH072 (VHH-I2S), or rAAV9-GTH069 (I2S-VHH) at 2.5×10¹² vg/kg.

FIGS. 7A-7B are graphical representations of sustained hI2S activity in serum throughout 4-week study in IdsKO mice treated with gene therapy constructs expressing I2S with a liver-specific (FIG. 7A) or ubiquitous promoter (FIG. 7B).

FIGS. 8A-8B are graphical representations of GCB activity (nmol/hr/mg) from cell lysates (FIG. 8A) and supernatants (FIG. 8B) following transfection of plasmids expressing tagged and untagged GCB in Huh7 cells.

FIGS. 9A-9C are graphical representations of GCB activity (nmol/hr/mg; FIG. 9A), GL-1 levels (normalized total GL1, ng/mg protein; FIG. 9B), and lyso-GL1 levels (normalized lysoGL1, ng/mg protein; FIG. 9C) in the brain of D409V mice after injection of buffer, pGTG077, or pGTG072.

FIGS. 10A-10C show graphs of GCB activity (nmol/hr/ml) in serum (FIG. 10A), liver (FIG. 10B), and spleen (FIG. 10C) after injection of buffer, pGTG077, or pGTG072 in D409V mice.

FIG. 11 is a graphical representation of percent SGSH activity following transfection of plasmids expressing tagged and untagged SGSH in Huh7 cells.

FIGS. 12A-12C are graphical representations of SGSH concentration in brain (ng of hSGSH/mg of total; FIG. 12A), serum (ng/mL; FIG. 12B), and liver (ng of SGSH/mg of total; FIG. 12C) in WT mice after injection of vehicle, SGSH, SGSH-BBB1, or BBB1-SGSH, as indicated.

FIG. 13 shows the brain exposure to serum ratio of SGSH in WT mice after injection of SGSH, SGSH-BBB1, or BBB1-SGSH.

FIGS. 14A-14D are schematic representations of example vector constructs of the present disclosure. FIGS. 14A-14B show constructs comprising an IDS transgene sequence under the control of a ubiquitous promoter (“Ubiq”; FIG. 14A) or a liver-specific promoter (“LSP”) and a liver-specific enhancer (“LSE”; FIG. 14B). FIGS. 14C-14D show constructs comprising a SGSH transgene sequence under the control of a liver-specific promoter (“LSP”) and a liver-specific enhancer (“LSE”). SP=signal peptide.

FIGS. 15A-15B are bar graphs illustrating the percent of SGSH protein measured crossing an in vitro Mimetas system membrane at high (FIG. 15A) and low (FIG. 15B) concentrations of SGSH BBB1 fusion constructs.

FIGS. 16A-16E are graphical representations of transcytosis as measured using a transwell model. FIG. 16A is a representative image of TEER value after 3d post seeding with hBMEC cells compared to cell free wells. FIGS. 16B-16E are bar graphs illustrating SGSH activity (FIG. 16B) and transcytosed SGSH protein (FIG. 16C) using transfected media and transcytosed SGSH protein (FIG. 16D) and SGSH activity (FIG. 16E) using purified protein.

FIGS. 17A-17C are bar graphs showing the concentration of GAG in cultured fibroblasts compared to normal cells (FIG. 17A) and dose dependent accumulation of SGSH in fibroblasts at 3 days (FIG. 17B) and 5 days (FIG. 17C) of culture.

FIGS. 18A-18J are representative images of control (FIGS. 18A-18D) and MPSIIIA patient-derived (FIGS. 18E-18J) fibroblasts stained with Lysotracker red (FIGS. 18A, 18C, 18E, 18G, and 18I) and Hoechst (FIGS. 18B, 18D, 18F, 18H, and 18J).

FIGS. 19A-19D are bar graphs illustrating liver (FIG. 19A), serum (FIG. 19B), and brain (FIG. 19C) SGSH levels and relative brain SGSH activity (FIG. 19D) in wild-type mouse administered control and SGSH BBB1 fusion constructs by hydrodynamic tail vain injection.

FIGS. 20A-20F are sample images of immuno-histochemistry for SGSH in brain samples obtained from wild-type mice administered SGSH C-terminal (FIGS. 20C-20D) and N-terminal (FIGS. 20E-20F) fusion constructs by hydrodynamic tail vain injection, as compared to mice administered a control SGSH.

FIGS. 21A-21G show characteristics of an MPSIIIA mouse model. FIGS. 21A-21D show brain (FIG. 21A), kidney (FIG. 21), liver (FIG. 21C), and spleen (FIG. 21D) SGSH activity in wild type mice and mice heterologous or homozygous for a knock-in point mutation in the Sgsh gene. FIGS. 21E-21G show sample histological images of anti-LAMP1 staining in the cortex of wild type mice (FIG. 21E) and mice heterologous (FIG. 21F) or homozygous (FIG. 21G) for a knock-in point mutation in the Sgsh gene.

### DETAILED DESCRIPTION

Some aspects of the present disclosure are related to viral vectors comprising a transgene capable of crossing the blood brain barrier and uses thereof in the treatment of diseases presenting with CNS manifestations. Diseases that affect the CNS can be very difficult to treat, especially when the mechanism of the disease involves a defective or missing protein, e.g., an enzyme. This is the case for various lysosomal storage disorders (LSDs), many of which are characterized by a defect in a single lysosomal enzyme. Administration of recombinant enzyme replacement therapy (ERT) remains the standard of care for various LSDs; however, these enzymes do not readily cross the blood brain barrier, significantly limiting their effect on LSD patients. Further, these therapies must be delivered periodically in order to maintain levels of substrate reduction necessary to alleviate symptoms. As such, the compositions and methods disclosed herein provide a novel and effective means for delivering recombinant proteins (e.g., enzymes) across the blood brain barrier and reducing and/or ameliorating the devastating CNS manifestations of various LSDs, including, but not limited to mucopolyscahharidoses such as Hurler syndrome (MPS I), Hunter syndrome (MPS II), Sanfilippo syndrome (MPS III), Sly syndrome (MPS VII), Gaucher disease, Metachromatic leukodystrophy, Krabbe disorder, and GM1 gangliosidosis.

The compositions and methods described herein allow for increased localization across the BBB of proteins that correct a cellular dysfunction within the CNS, e.g., a lysosomal enzyme which is otherwise deficient in case of an LSD. These compositions further utilize viral vector gene therapy as a means of providing continuous expression of the protein in the subject, thereby obviating the need for repetitive re-dosing, and providing patients with lasting substrate reduction and alleviation of disease symptoms.

### I. Definitions

As used herein, the term “about” or “approximately”, as applied to one or more values of interest, refers to a value that is similar to a stated reference value. In certain aspects, the term “about” or “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). It is understood that when the term “about” or “approximately” is used to modify a stated reference value, the stated reference value itself is covered along with values that are near the stated reference value on either side of the stated reference value.

As used herein, the term “active” or “activity” refers to forms of a therapeutic protein, which retain a biological activity of the corresponding native or naturally occurring polypeptide. The activity may be greater than, equal to, or less than that observed with the corresponding native or naturally occurring polypeptide.

As used herein, the terms “administer”, “administration”, and “administering” refer to providing a composition of the present disclosure to a subject in need thereof (e.g., to a person suffering from the effects of Hunter disease). The composition can be administered by any route. In some aspects, the composition is administered by intravenous administration, intraperitoneal administration, intraocular administration, oral administration, inhalation, intrathecal administration, intracranial administration, intracarotid artery, intra-cisterna magna (ICM), intracerebroventricular (ICV), intra-arterial administration, or any combination thereof.

As used herein, the term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

As used herein, the term “amino acid substitution” refers to replacing an amino acid residue present in a parent or reference sequence with another amino acid residue. An amino acid can be substituted in a parent or reference sequence, for example, via chemical peptide synthesis or through recombinant methods known in the art. Accordingly, a reference to a “substitution at position X” refers to the substitution of an amino acid present at position X in a reference sequence with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residues.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}).

Substitutions in a protein or polypeptide amino acid sequence may either be conservative or non-conservative in nature. A conservative amino acid substitution refers to a substitution of a residue with a different residue having a similar side chain, and thus typically involves substitution of the amino acid in a polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. A non-conservative substitution refers to substitution of an amino acid in a polypeptide with an amino acid with significantly differing side chain properties. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

In some aspects, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “animal” refers to any member of the animal kingdom. In some aspects, “animal” refers to humans at any stage of development. In some aspects, “animal” refers to non-human animals at any stage of development. In certain aspects, the non-human animal is a mammal (e.g., a rodent, a mouse, a rat, a rabbit, a monkey, a dog, a cat, a sheep, cattle, a primate, or a pig). In some aspects, animals include, but are not limited to, mammals, birds, reptiles, amphibians, fish, and worms. In some aspects, the animal is a transgenic animal, genetically-engineered animal, or a clone.

As used herein, the term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within a variable region of the immunoglobulin molecule. As used herein, the term “antibody” encompasses polyclonal antibodies, monoclonal antibodies, antibody fragments (such as Fab, Fab′, F(ab′)2, and Fv fragments), single chain Fv (scFv) mutants, multispecific antibodies such as bispecific antibodies for example generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The different classes of immunoglobulins have different and well-known subunit structures and three-dimensional configurations. Antibodies can be naked or conjugated to other molecules such as toxins, radioisotopes, etc.

A “VHH”, as used herein, refers to variable domain of a heavy chain-only VHH molecule. In some aspects, the VHH is a camelid heavy chain-only VHH molecule. In some aspects, the VHH is generated from a human antibody, a humanized antibody, or a synthetic antibody. A VHH is generally around 15 kDa in size, and it contains a single chain molecule that can bind its cognate antigen using a single domain. VHHs typically comprise four framework regions (FRs) and three complementarity determining regions (or CDRs), which have high variability both in sequence content and structure conformation and are involved in antigen binding and provide antigen specificity. In some aspects, the VHH is modified to enhance affinity, stability, solubility, and/or resistance to aggregation. For example, compared to a conventional human antibody VH, one or more amino acids in the FR2 region and CDRs of the VHH can be substituted in the FR2 region and complementarity-determining regions (CDRs) of the VHH. In some aspects, one or more highly conserved hydrophobic amino acids (e.g., Val47, Gly49, Leu50, and/or Trp52) in the FR2 region are replaced by hydrophilic amino acids (e.g., Phe42, Glu49, Arg50, Gly52), rendering the overall structure more hydrophilic and contributing to high stability, solubility and resistance to aggregation.

As used herein, the terms “codon substitution” or “codon replacement” in the context of sequence optimization refer to replacing a codon present in a reference nucleic acid sequence with another codon. A codon can be substituted in a reference nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

As used herein, the term “codon-optimized” or “codon optimization” refers to changes in the codons of the polynucleotide encoding a protein such that the encoded protein is more efficiently expressed, e.g., in a cell or an organism. In some aspects, a nucleotide sequence disclosed herein, e.g., a transgene, is codon-optimized for expression in a human cell, e.g., in vivo.

The term “gene”, as used herein, refers to a DNA region encoding a protein or polypeptide, as well as all DNA regions which regulate the production of the protein or polypeptide, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, the term “transgene” refers to a heterologous DNA region encoding a polypeptide. A transgene does not necessarily include promoter sequences, terminators, translational regulatory sequences such as ribosome binding sites and internal ribosome entry sites, enhancers, silencers, insulators, boundary elements, replication origins, matrix attachment sites and locus control regions. In some aspects, a transgene consists of a polypeptide coding region. In some embodiments, a transgene comprises a nucleic acid encoding a biologically active polypeptide (e.g., an enzyme) linked to a nucleic acid encoding a tag (e.g., a nucleic acid encoding an antigen binding molecule). In some embodiments, the transgene comprises a nucleic acid molecule encoding a biologically active polypeptide linked to a nucleic acid molecule encoding a tag, where there is a linker sequence between the nucleic acid encoding the polypeptide and the nucleic acid encoding the tag. In some embodiments, the nucleic acid encoding the tag is 5′ of the nucleic acid encoding the polypeptide. In other embodiments, the nucleic acid encoding the tag is 3′ of the nucleic acid encoding the polypeptide.

The term “genome particles (gp)” or “genome equivalents”, as used herein in reference to a viral titer, refers to the number of virions containing the recombinant AAV (rAAV) DNA genome, regardless of infectivity or functionality.

The terms “idursulfase”, “IDS”, “iduronate-2-sulfatase”, and “I2S”, are used interchangeably to refer to a lysosomal enzyme involved in the degradation pathway of dermatan sulfate and heparan sulfate (see UniProtKB-P22304).

The terms “Lysosomal acid glucosylceramidase”, “beta-glucocerebrosidase”, “GBA”, “acid beta-glucosidase”, “Cholesterol glucosyltransferase”, “Cholesteryl-beta-glucosidase”, “D-glucosyl-N-acylsphingosine glucohydrolase” and “GCB”, are used interchangeably to refer to a lysosomal enzyme involved in the degradation pathway of glucosylceramide into free ceramide and glucose (see UniProtKB—P04062).

The terms “N-sulphoglucosamine sulphohydrolase”, “Sulfoglucosamine sulfamidase”, “SGSG”, “HNS”, “Sulphamidase”, are used interchangeably to refer to a lysosomal enzyme involved in the degradation pathway of lysosomal heparan sulfate (see UniProtKB—P51688).

As used herein, the term “linker” refers to any molecule or bond that connects two or more moieties. In some aspects, the linker is a peptide linker, e.g., the linker comprises one or more peptide bonds. In some aspects, the peptide linker comprises one or more peptides, e.g., a polypeptide. In some aspects, the linker is a chemical linker.

As used herein, the term “nervous system” includes both the central nervous system and the peripheral nervous system. The term “central nervous system” or “CNS” includes all cells and tissue of the brain and spinal cord of a vertebrate. The term “peripheral nervous system” refers to all cells and tissue of the portion of the nervous system outside the brain and spinal cord. Thus, the term “nervous system” includes, but is not limited to, neuronal cells, glial cells, astrocytes, cells in the cerebrospinal fluid (CSF), cells in the interstitial spaces, cells in the protective coverings of the spinal cord, epidural cells (i.e., cells outside of the dura mater), cells in non-neural tissues adjacent to or in contact with or innervated by neural tissue, cells in the epineurium, perineurium, endoneurium, funiculi, fasciculi, and the like.

As used herein, the terms “nucleic acid”, “polynucleotide”, and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

As used herein, the terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

The terms “polypeptide” and “protein”, used interchangeably herein, or a nucleotide sequence encoding the same, refer to a protein or nucleotide sequence, respectively, that represents either a native sequence, a variant thereof or a fragment thereof. The full-length proteins, with or without the signal sequence, and fragments thereof, as well as proteins with modifications, such as deletions, additions and substitutions (either conservative or non-conservative in nature), to the native sequence, are intended for use herein, so long as the protein maintains the desired activity. These modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the desired activity of the native molecule, are contemplated for use herein.

As used herein, the term “promoter” encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. In some aspects, liver-specific promoters include, for example, transthyretin promoter (TTR); thyroxine-binding globulin (TBG) promoter; hybrid liver-specific promoter (HLP), and alpha-1-antitrypsin (AAT) promoter. Promoters may be “constitutive”, meaning continually active, or “inducible”, meaning the promoter can be activated or deactivated by the presence or absence of biotic or abiotic factors. Also included in the nucleic acid constructs or vectors of the present disclosure are enhancer sequences that may or may not be contiguous with the promoter sequence. Enhancer sequences influence promoter-dependent gene expression and may be located in the 5′ or 3′ regions of the native gene. In some aspects, the enhancer is a tissue specific enhancer. In some aspects, the enhancer is a liver-specific enhancer. In certain aspects, the construct comprises a liver-specific enhancer and a liver-specific promoter upstream of the transgene.

As used herein, the term “sequence optimization” refers to a process or series of processes by which nucleobases in a reference nucleic acid sequence are replaced with alternative nucleobases, resulting in a nucleic acid sequence with improved properties, e.g., improved protein expression or increased activity.

The terms “subject”, “individual”, and “patient” are used interchangeably herein and refer to a vertebrate, preferably a mammal. Mammals include, but are not limited to, murines, rodents, simians, humans, farm animals, sport animals and pets.

The term “TAG” or “tag”, as used herein, refers to a polypeptide that facilitates targeted localization of an associated second polypeptide to which it is linked. In some aspects, the TAG comprises an antibody or an antigen-binding portion thereof that specifically binds human transferrin receptor 1 (TfR1). In some aspects, the TAG comprises a VHH that specifically binds human TfR1. In some embodiments, a transgene contained in a viral vector encompassed by the disclosure herein comprises a nucleic acid encoding a polypeptide (e.g., an enzyme which is mutated or missing in a LSD) linked (directly or indirectly (e.g., via a linker nucleic acid sequence)) to a nucleic acid encoding a TAG. In some embodiments, the tag is N-terminus of the polypeptide. In other embodiments, the tag is C-terminus of the polypeptide. In yet other embodiments, the tag is present both N- and C-termini of the polypeptide. In some embodiments, more than one tag may be linked (directly or indirectly via a linker) to a polypeptide.

The term “therapeutic”, “effective amount”, or “therapeutically effective amount” of a composition or agent, as provided herein, refer to a sufficient amount of the composition or agent to provide the desired response, such as the prevention, delay of onset, or amelioration of symptoms in a subject or an attainment of a desired biological outcome.

The term “transferrin receptor” or “TfR” refers to a type II homodimeric transmembrane glycoprotein consisting of two identical 90 kDa subunits linked by two disulfide bridges (Jing and Trowbridge, EMBO J. 6(2):327-31 (1987); McClelland et al., Cell 39(2):267-74 (1984)). Each monomer has a short cytoplasmic N-terminal domain of 61 amino acids containing a YTRF (Tyrosine-Threonine-Arginine-Phenylalanine) internalization motif, a single hydrophobic transmembrane segment of 27 amino acids, and a broad C-terminal extracellular domain of 670 amino acids, containing a trypsin cleavage site and a transferrin binding site. Each subunit is capable of binding a transferrin molecule. The extracellular domain has one O-glycosylation site and three N-glycosylation sites, the latter being particularly important for the proper folding and transport of the receptor to the cell surface. There are also palmitylation sites in the intramembranous domain, that may anchor the receptor and allow its endocytosis. In addition, an intracellular phosphorylation site is present, which has unknown functions, and which plays no role in endocytosis.

As used herein, the term “vector” refers to a nucleic acid molecule that is capable of transferring gene sequences to target cells. Typically, “vector construct”, “expression vector”, and “gene transfer vector”, mean any nucleic acid construct capable of directing the expression of a gene of interest, and which can transfer gene sequences to target cells. Thus, the term includes cloning, and expression vehicles, as well as integrating vectors. In some aspects, the vector is a virus, which includes, for example, encapsulated forms of vector nucleic acids, and viral particles in which the vector nucleic acids have been packaged. In some aspects, the vector is not a wild-type strain of a virus, in as much as it comprises human-made mutations or modifications. In some aspects, the vector is derived from a wild-type viral strain by genetic manipulation (i.e., by deletion) to comprise a conditionally replicating virus, as further described herein. In some aspects, the vector is delivered by non-viral means. In some aspects, vectors described herein are gene therapy vectors, which are used as carriers for delivery of polynucleotide sequences to cells. In some embodiments, a gene therapy vector is a viral vector. In other embodiments, a gene therapy vector is a non-viral vector. In a particular aspect, a gene therapy vector described herein is a recombinant AAV vector (e.g., AAV8 or AAV9).

The recitation of numerical ranges by endpoints herein includes all numbers and fractions subsumed within that range (e.g., 1 to 5 includes 1, 1.5, 2, 2.75, 3, 3.9, 4 and 5). It is also to be understood that all numbers and fractions thereof are presumed to be modified by the term “about.”

Various aspects of the disclosure are described in detail in the following sections. The use of sections is not meant to limit the disclosure. Each section can apply to any aspect of the disclosure. In this application, the use of “or” means “and/or” unless stated otherwise. As used herein, the singular forms “a”, “an”, and “the” include both singular and plural referents unless the context clearly dictates otherwise.

### II. Compositions of the Disclosure

Some aspects of the present disclosure are directed to a viral vector comprising a promoter and a transgene, wherein the transgene comprises (i) a nucleotide sequence encoding a biologically active polypeptide and (ii) a nucleotide sequence encoding a TAG. In some aspects, the viral vector comprises an adeno-associated virus, a lentivirus, a retrovirus, a variant thereof, or a combination thereof. Some aspects of the present disclosure are directed to a recombinant AAV (rAAV) vector comprising (a) a 5′ inverted terminal repeat (ITR), (b) a promoter, (c) a transgene comprising (i) a nucleotide sequence encoding a biologically active polypeptide and (ii) a nucleotide sequence encoding a TAG, and (d) a 3′ ITR. In some aspects, the TAG increases the translocation of the biologically active polypeptide across the blood brain barrier. In some embodiments, a linker nucleic acid sequence is present between (i) and (ii).

In some aspects, the transgene comprises, in 5′ to 3′ order (i) a nucleotide sequence encoding a biologically active polypeptide and (ii) a nucleotide sequence encoding a TAG. As such, in some aspects, the nucleotide sequence encoding the biologically active polypeptide is 5′ of the nucleotide sequence encoding the TAG. In some aspects, the transgene comprises, in 5′ to 3′ order (i) a nucleotide sequence encoding a TAG and (ii) a nucleotide sequence encoding a biologically active polypeptide. As such, in some aspects, the nucleotide sequence encoding the biologically active polypeptide is 3′ of the nucleotide sequence encoding the TAG. In some embodiments, a linker nucleic acid sequence is present between (i) and (ii).

In some aspects, the nucleotide sequence encoding the biologically active polypeptide is linked to the nucleotide sequence encoding the TAG by a nucleotide sequence encoding a peptide linker. In some aspects, the linker is a flexible linker, a cleavable linker, a processable linker, or any combination thereof. In some aspects, the linker is a flexible linker. In some aspects, the linker is a cleavable linker.

### A. Biologically Active Polypeptides

In some aspects, the biologically active polypeptide comprises a therapeutic enzyme, an antibody or an antigen-binding portion thereof, a growth factor, a hormone, a cytokine, a chemokine, an inhibitory ligand, an agonistic ligand, or any combination thereof. In some aspects, the biologically active polypeptide comprises a protein that corrects a cellular dysfunction, e.g., in the CNS. In some aspects, the biologically active polypeptide comprises a therapeutic enzyme. In some embodiments, the biologically active polypeptide comprises an enzyme that is otherwise mutated or missing in case of a metabolic disorder such as a lysosomal storage disease. In some aspects, the biologically active polypeptide has an activity, binds a target, interacts with a receptor, interacts with a ligand, catalyzes a reaction, or acts as a substrate in the central nervous system (CNS).

In some aspects, the biologically active polypeptide comprises a therapeutic enzyme. In some aspects, the therapeutic enzyme has an activity in the CNS. In some aspects, the therapeutic enzyme catalyzes the processing of a substrate in the CNS, thereby reducing the level of substrate in the CNS. In some aspects, the therapeutic enzyme has an activity in the CNS and in one or more other parts of the body. In some aspects, the therapeutic enzyme catalyzes the processing of a substrate in the CNS and in one or more other parts of the body, thereby reducing the level of substrate.

In some aspects, the biologically active polypeptide has an idursulfase activity. In some aspects, the biologically active polypeptide is an idursulfase (I2S) or a functional variant thereof. In some aspects, the biologically active polypeptide comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 1, wherein the biologically active polypeptide has an idursulfase activity. In some aspects, the biologically active polypeptide comprises the amino acid sequence set forth in SEQ ID NO: 1.

In some aspects, the nucleotide sequence encoding the biologically active polypeptide comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 2, wherein the biologically active polypeptide encoded by the nucleotide sequence has an idursulfase activity. In some aspects, the nucleotide sequence encoding the biologically active polypeptide comprises the nucleic acid sequence set forth in SEQ ID NO: 2.

In some aspects, the nucleotide sequence encoding the biologically active polypeptide is codon-optimized. In some aspects, the nucleotide sequence encoding the biologically active polypeptide is codon-optimized for human in vivo expression. In some aspects, the codon-optimized nucleotide sequence encoding the biologically active polypeptide comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 3 or 4, wherein the biologically active polypeptide encoded by the nucleotide sequence has an idursulfase activity. In some aspects, the codon-optimized nucleotide sequence encoding the biologically active polypeptide comprises the nucleic acid sequence set forth in SEQ ID NO: 3 or 4.

TABLE 1
Transgene Sequences

| IDS Amino | MPPPRTGRGLLWLGLVLSSVCVALGSETQANSTTDALNVLLIIVDDLRPSLGCYGDKLVR |
|---|---|
| Acid | SPNIDQLASHSLLFQNAFAQQAVCAPSRVSFLTGRRPDTTRLYDFNSYWRVHAGNFSTIP |
| Sequence | QYFKENGYVTMSVGKVFHPGISSNHTDDSPYSWSFPPYHPSSEKYENTKTCRGPDGELHA |
| | NLLCPVDVLDVPEGTLPDKQSTEQAIQLLEKMKTSASPFFLAVGYHKPHIPFRYPKEFQK |
| | LYPLENITLAPDPEVPDGLPPVAYNPWMDIRQREDVQALNISVPYGPIPVDFQRKIRQSY |
| | FASVSYLDTQVGRLLSALDDLQLANSTIIAFTSDHGWALGEHGEWAKYSNFDVATHVPLI |
| | FYVPGRTASLPEAGEKLFPYLDPFDSASQLMEPGRQSMDLVELVSLFPTLAGLAGLQVPP |
| | RCPVPSFHVELCREGKNLLKHFRFRDLEEDPYLPGNPRELIAYSQYPRPSDIPQWNSDKP |
| | SLKDIKIMGYSIRTIDYRYTVWVGFNPDEFLANFSDIHAGELYFVDSDPLQDHNMYNDSQ |
| | GGDLFQLLMP (SEQ ID NO: 1) |
| IDS Nucleic | ATGCCGCCACCCCGGACCGGCCGAGGCCTTCTCTGGCTGGGTCTGGTTCTGAGCTCCGTC |
| Acid Coding | TGCGTCGCCCTCGGATCCGAAACGCAGGCCAACTCGACCACAGATGCTCTGAACGTTCTT |
| Sequence | CTCATCATCGTGGATGACCTGCGCCCCTCCCTGGGCTGTTATGGGGATAAGCTGGTGAGG |
| | TCCCCAAATATTGACCAACTGGCATCCCACAGCCTCCTCTTCCAGAATGCCTTTGCGCAG |
| | CAAGCAGTGTGCGCCCCGAGCCGCGTTTCTTTCCTCACTGGCAGGAGACCTGACACCACC |
| | CGCCTGTACGACTTCAACTCCTACTGGAGGGTGCACGCTGGAAACTTCTCCACCATCCCC |
| | CAGTACTTCAAGGAGAATGGCTATGTGACCATGTCGGTGGGAAAAGTCTTTCACCCTGGG |
| | ATATCTTCTAACCATACCGATGATTCTCCGTATAGCTGGTCTTTTCCACCTTATCATCCT |
| | TCCTCTGAGAAGTATGAAAACACTAAGACATGTCGAGGGCCAGATGGAGAACTCCATGCC |
| | AACCTGCTTTGCCCTGTGGATGTGCTGGATGTTCCCGAGGGCACCTTGCCTGACAAACAG |
| | AGCACTGAGCAAGCCATACAGTTGTTGGAAAAGATGAAAACGTCAGCCAGTCCTTTCTTC |
| | CTGGCCGTTGGGTATCATAAGCCACACATCCCCTTCAGATACCCCAAGGAATTTCAGAAG |
| | TTGTATCCCTTGGAGAACATCACCCTGGCCCCCGATCCCGAGGTCCCTGATGGCCTACCC |
| | CCTGTGGCCTACAACCCCTGGATGGACATCAGGCAACGGGAAGACGTCCAAGCCTTAAAC |
| | ATCAGTGTGCCGTATGGTCCAATTCCTGTGGACTTTCAGCGGAAAATCCGCCAGAGCTAC |
| | TTTGCCTCTGTGTCATATTTGGATACACAGGTCGGCCGCCTCTTGAGTGCTTTGGACGAT |
| | CTTCAGCTGGCCAACAGCACCATCATTGCATTTACCTCGGATCATGGGTGGGCTCTAGGT |
| | GAACATGGAGAATGGGCCAAATACAGCAATTTTGATGTTGCTACCCATGTTCCCCTGATA |
| | TTCTATGTTCCTGGAAGGACGGCTTCACTTCCGGAGGCAGGCGAGAAGCTTTTCCCTTAC |
| | CTCGACCCTTTTGATTCCGCCTCACAGTTGATGGAGCCAGGCAGGCAATCCATGGACCTT |
| | GTGGAACTTGTGTCTCTTTTTCCCACGCTGGCTGGACTTGCAGGACTGCAGGTTCCACCT |
| | CGCTGCCCCGTTCCTTCATTTCACGTTGAGCTGTGCAGAGAAGGCAAGAACCTTCTGAAG |
| | CATTTTCGATTCCGTGACTTGGAAGAGGATCCGTACCTCCCTGGTAATCCCCGTGAACTG |
| | ATTGCCTATAGCCAGTATCCCCGGCCTTCAGACATCCCTCAGTGGAATTCTGACAAGCCG |
| | AGTTTAAAAGATATAAAGATCATGGGCTATTCCATACGCACCATAGACTATAGGTATACT |
| | GTGTGGGTTGGCTTCAATCCTGATGAATTTCTAGCTAACTTTTCTGACATCCATGCAGGG |
| | GAACTGTATTTTGTGGATTCTGACCCATTGCAGGATCACAATATGTATAATGATTCCCAA |
| | GGTGGAGATCTTTTCCAGTTGTTGATGCCTTGA (SEQ ID NO: 2) |
| Codon- | ATGCCACCCC CCCGGACCGG GAGAGGCCTC TTGTGGTTGG GCCTGGTGCT |
| Optimized | GAGCAGCGTG TGCGTGGCCC TGGGCAGTGA GACCCAGGCT AACTCTACAA |
| I2S Nucleic | CAGATGCCTT GAATGTGCTG CTGATCATTG TGGATGACCT GAGGCCAAGT |
| Acid | CTGGGCTGCT ATGGGGACAA ATTGGTGAGG TCCCCCAACA TCGACCAGTT |
| Sequence-1 | GGCCTCCCAC TCTCTCCTAT TCCAAAATGC TTTCGCCCAG CAGGCAGTTT |
| | GTGCCCCCTC TAGGGTGAGC TTCCTCACTG GCAGGCGCCC TGACACCACT |
| | AGACTGTATG ACTTTAACAG CTATTGGAGG GTGCACGCAG GAAACTTCTC |
| | CACAATCCCT CAATACTTCA AGGAGAATGG TTATGTGACA ATGTCTGTGG |
| | GCAAGGTGTT CCACCCTGGC ATCAGCAGCA ACCACACCGA TGACTCACCC |
| | TATAGTTGGT CTTTTCCCCC CTACCATCCT TCATCTGAGA AATATGAAAA |
| | CACAAAAACC TGCCGAGGCC CAGACGGGGA ACTGCATGCC AACCTACTCT |
| | GTCCTGTTGA TGTACTGGAC GTGCCCGAGG GCACCCTCCC TGATAAGCAG |
| | TCCACAGAAC AGGCCATTCA GCTGCTTGAA AAGATGAAGA CCTCCGCATC |
| | CCCCTTCTTC TTGGCTGTCG GCTACCACAA GCCCCATATC CCCTTTAGAT |
| | ACCCCAAGGA ATTCCAGAAA CTGTACCCAC TGGAGAACAT CACACTTGCT |
| | CCTGACCCTG AAGTGCCTGA CGGACTGCCT CCAGTGGCCT ATAACCCTTG |
| | GATGGACATC CGGCAGCGCG AGGATGTGCA GGCTCTGAAC ATTAGTGTGC |
| | CTTATGGGCC CATCCCTGTG GACTTTCAGA GGAAGATTCG CCAGTCCTAC |
| | TTTGCCTCTG TATCCTACCT GGACACACAG GTGGGACGCC TGCTGTCTGC |
| | CCTTGATGAT CTGCAACTGG CCAACAGCAC CATTATAGCT TTCACATCAG |
| | ACCATGGGTG GGCTCTTGGG GAGCATGGTG AATGGGCTAA GTACTCCAAC |
| | TTCGATGTGG CAACCCATGT CCCTCTGATC TTCTATGTGC CAGGAAGGAC |
| | CGCCTCTCTG CCAGAGGCAG GTGAGAAGCT GTTCCCCTAT CTGGACCCTT |
| | TTGACTCCGC CAGCCAGCTG ATGGAGCCTG GCCGACAGTC TATGGACCTG |
| | GTTGAGCTGG TCAGCCTGTT TCCCACACTC GCTGGACTGG CTGGCCTGCA |
| | AGTACCCCCA CGCTGCCCAG TGCCCTCCTT CCATGTGGAG CTTTGCAGGG |
| | AGGGGAAGAA CCTCCTCAAG CACTTCAGGT TCAGGGACCT AGAGGAGGAT |
| | CCTTATCTGC CTGGAAACCC CAGAGAGCTT ATTGCTTACT CCCAGTATCC |
| | AAGGCCTAGT GACATTCCCC AATGGAACTC AGACAAACCA AGCCTGAAAG |
| | ACATCAAGAT CATGGGATAC TCTATCAGGA CCATTGACTA CAGGTACACT |
| | GTGTGGGTTG GCTTCAACCC GGATGAGTTC CTGGCTAATT TCTCTGACAT |
| | ACATGCTGGC GAGCTGTACT TCGTGGACAG TGACCCCCTG CAGGATCACA |
| | ACATGTACAA TGATTCCCAG GGGGGTGACC TCTTCCAGCT TCTGATGCCC |
| | TAA (SEQ ID NO: 3) |
| Codon- | ATGCCACCAC CTAGGACAGG CAGGGGCCTG CTTTGGCTTG GACTGGTGCT |
| Optimized | GAGCTCTGTC TGTGTTGCCC TGGGCTCCGA GACCCAAGCC AACTCTACAA |
| I2S Nucleic | CCGATGCTCT CAATGTTCTG CTCATCATAG TGGATGACCT GCGGCCCTCT |
| Acid | CTAGGCTGCT ATGGAGACAA GTTGGTGCGG AGCCCCAACA TAGACCAGCT |
| Sequence-2 | AGCCTCTCAC TCCCTGCTGT TCCAGAATGC CTTCGCCCAG CAAGCTGTGT |
| | GCGCCCCCTC TAGAGTGTCT TTCCTGACCG GGAGAAGGCC TGATACAACA |
| | AGGCTGTATG ACTTTAACAG CTACTGGAGG GTGCACGCAG GCAACTTCTC |
| | CACTATCCCC CAATACTTCA AGGAGAATGG CTATGTGACC ATGAGCGTGG |
| | GCAAGGTCTT CCACCCTGGA ATCTCCTCCA ACCACACTGA TGATAGTCCC |
| | TACTCTTGGT CTTTTCCTCC CTATCACCCT AGCAGTGAGA AGTATGAGAA |
| | CACCAAAACC TGCAGAGGCC CTGATGGGGA GCTGCATGCT AACCTCCTGT |
| | GTCCTGTAGA TGTGCTGGAC GTCCCAGAGG GCACCTTGCC AGATAAGCAG |
| | TCTACTGAGC AGGCTATCCA GCTGCTTGAG AAAATGAAGA CTTCTGCATC |
| | TCCCTTCTTT CTGGCTGTTG GCTACCACAA GCCTCACATC CCCTTCAGGT |
| | ACCCTAAGGA GTTCCAAAAG CTCTATCCTC TGGAAAACAT CACACTTGCC |
| | CCCGATCCTG AGGTCCCTGA CGGCCTCCCA CCAGTAGCCT ACAATCCTTG |
| | GATGGACATT AGGCAGAGAG AGGATGTCCA GGCTCTGAAT ATTTCTGTGC |
| | CCTATGGGCC CATCCCGGTG GACTTCCAGC GCAAAATCAG ACAGTCCTAC |
| | TTTGCCTCTG TGAGCTATCT GGACACCCAG GTTGGGAGGC TCCTCTCCGC |
| | CCTTGACGAC CTCCAGTTGG CCAACAGCAC CATTATAGCC TTCACCTCTG |
| | ACCACGGCTG GGCACTGGGG GAACACGGGG AGTGGGCTAA GTACTCTAAC |
| | TTTGATGTGG CCACCCACGT GCCCCTCATC TTTTATGTGC CTGGCAGGAC |
| | TGCCAGCCTG CCCGAAGCTG GGGAAAAACT GTTTCCATAC CTGGACCCTT |
| | TTGACAGTGC TTCTCAGCTC ATGGAACCTG GCCGTCAGAG CATGGATCTG |
| | GTGGAGCTAG TGTCCCTCTT CCCAACCTTG GCTGGCCTTG CTGGTCTCCA |
| | GGTGCCTCCT AGATGCCCAG TCCCCTCCTT CCATGTTGAA CTCTGCCGTG |
| | AGGGGAAGAA TCTGCTGAAG CACTTCAGAT TCAGAGACTT GGAGGAGGAC |
| | CCCTACCTTC CTGGGAACCC CAGGGAGTTG ATTGCATACT CCCAGTATCC |
| | CAGGCCAAGT GACATTCCCC AGTGGAACTC CGACAAACCA AGTCTGAAGG |
| | ACATCAAGAT CATGGGGTAC AGCATCAGGA CCATTGACTA CAGATACACA |
| | GTGTGGGTTG GATTTAACCC AGATGAGTTC TTGGCAAACT TTTCTGACAT |
| | CCATGCAAGT CAGTTGTATT TTGTGGACAG CGACCCTCTG CAGGATCACA |
| | ACATGTACAA TGACAGCCAG GGTGGGGACC TCTTTCAACT CCTCATGCCA |
| | TAG (SEQ ID NO: 4) |
| GCB | MEFSSPSREECPKPLSRVSIMAGSLTGLLLLQAVSWASGARPCIPKSFGYSSVVCVCNAT |
| (hGBA1) | YCDSFDPPTFPALGTFSRYESTRSGRRMELSMGPIQANHTGTGLLLTLQPEQKFQKVKGF |
| Amino Acid | GGAMTDAAALNILALSPPAQNLLLKSYFSEEGIGYNIIRVPMASCDFSIRTYTYADTPDD |
| Sequence | FQLHNFSLPEEDTKLKIPLIHRALQLAQRPVSLLASPWTSPTWLKTNGAVNGKGSLKGQP |
| | GDIYHQTWARYFVKFLDAYAEHKLQFWAVTAENEPSAGLLSGYPFQCLGFTPEHQRDFIA |
| | RDLGPTLANSTHHNVRLLMLDDQRLLLPHWAKVVLTDPEAAKYVHGIAVHWYLDFLAPAK |
| | ATLGETHRLFPNTMLFASEACVGSKFWEQSVRLGSWDRGMQYSHSIITNLLYHVVGWTDW |
| | NLALNPEGGPNWVRNFVDSPIIVDITKDTFYKQPMFYHLGHFSKFIPEGSQRVGLVASQK |
| | NDLDAVALMHPDGSAVVVVLNRSSKDVPLTIKDPAVGFLETISPGYSIHTYLWRRQ |
| | (SEQ ID NO: 41) (signal peptide) |
| GCB | atggagttttcaagtccttccagagaggaatgtcccaagcctttgagtagggtaagcatc |
| (hGBA1) | atggctggcagcctcacaggattgcttctacttcaggcagtgtcgtgggcatcaggtgcc |
| Coding | cgcccctgcatccctaaaagcttcggctacagctcggtggtgtgtgtctgcaatgccaca |
| Sequence | tactgtgactcctttgaccccccgacctttcctgcccttggtaccttcagccgctatgag |
| | agtacacgcagtgggcgacggatggagctgagtatggggcccatccaggctaatcacacg |
| | ggcacaggcctgctactgaccctgcagccagaacagaagttccagaaagtgaagggattt |
| | ggaggggccatgacagatgctgctgctctcaacatccttgccctgtcaccccctgcccaa |
| | aatttgctacttaaatcgtacttctctgaagaaggaatcggatataacatcatccgggta |
| | cccatggccagctgtgacttctccatccgcacctacacctatgcagacacccctgatgat |
| | ttccagttgcacaacttcagcctcccagaggaagataccaagctcaagatacccctgatt |
| | caccgagccctgcagttggcccagcgtcccgtttcactccttgccagcccctggacatca |
| | cccacttggctcaagaccaatggagcggtgaatgggaaggggtcactcaagggacagccc |
| | ggagacatctaccaccagacctgggccagatactttgtgaagttcctggatgcctatgct |
| | gagcacaagttacagttctgggcagtgacagctgaaaatgagccttctgctgggctgttg |
| | agtggataccccttccagtgcctgggcttcacccctgaacatcagcgagacttcattgcc |
| | cgtgacctaggtcctaccctcgccaacagtactcaccacaatgtccgcctactcatgctg |
| | gatgaccaacgcttgctgctgccccactgggcaaaggtggtactgacagacccagaagca |
| | gctaaatatgttcatggcattgctgtacattggtacctggactttctggctccagccaaa |
| | gccaccctaggggagacacaccgcctgttccccaacaccatgctctttgcctcagaggcc |
| | tgtgtgggctccaagttctgggagcagagtgtgcggctaggctcctgggatcgagggatg |
| | cagtacagccacagcatcatcacgaacctcctgtaccatgtggtcggctggaccgactgg |
| | aaccttgccctgaaccccgaaggaggacccaattgggtgcgtaactttgttgacagtccc |
| | atcattgtagacatcaccaaggacacgttttacaaacagcccatgttctaccaccttggc |
| | cacttcagcaagttcattcctgagggctcccagagagtggggctggttgccagtcagaag |
| | aacgacctggacgcagtggcactgatgcatcccgatggctctgctgttgtggtcgtgcta |
| | aaccgctcctctaaggatgtgcctcttaccatcaaggatcctgctgtgggcttcctggag |
| | acaatctcacctggctactc (SEQ ID NO: 42) |
| SGSH | MSCPVPACCALLLVLGLCRARPRNALLLLADDGGFESGAYNNSAIATPHLDALARRSLLF |
| (hSGSH) | RNAFTSVSSCSPSRASLLTGLPQHQNGMYGLHQDVHHFNSFDKVRSLPLLLSQAGVRTGI |
| Amino Acid | IGKKHVGPETVYPFDFAYTEENGSVLQVGRNITRIKLLVRKFLQTQDDRPFFLYVAFHDP |
| Sequence | HRCGHSQPQYGTFCEKFGNGESGMGRIPDWTPQAYDPLDVLVPYFVPNTPAARADLAAQY |
| | TTVGRMDQGVGLVLQELRDAGVLNDTLVIFTSDNGIPFPSGRTNLYWPGTAEPLLVSSPE |
| | HPKRWGQVSEAYVSLLDLTPTILDWFSIPYPSYAIFGSKTIHLTGRSLLPALEAEPLWAT |
| | VFGSQSHHEVTMSYPMRSVQHRHFRLVHNLNFKMPFPIDQDFYVSPTFQDLLNRTTAGQP |
| | TGWYKDLRHYYYRARWELYDRSRDPHETQNLATDPRFAQLLEMLRDQLAKWQWETHDPWV |
| | CAPDGVLEEKLSPQCQPLHNEL (SEQ ID NO: 43) |
| SGSH | ATGAGCTGCCCCGTGCCCGCCTGCTGCGCGCTGCTGCTAGTCCTGGGGCTCTGCCGGGCG |
| (hSGSH) | CGTCCCCGGAACGCACTGCTGCTCCTCGCGGATGACGGAGGCTTTGAGAGTGGCGCGTAC |
| Coding | AACAACAGCGCCATCGCCACCCCGCACCTGGACGCCTTGGCCCGCCGCAGCCTCCTCTTT |
| Sequence | CGCAATGCCTTCACCTCGGTCAGCAGCTGCTCTCCCAGCCGCGCCAGCCTCCTCACTGGC |
| | CTGCCCCAGCATCAGAATGGGATGTACGGGCTGCACCAGGACGTGCACCACTTCAACTCC |
| | TTCGACAAGGTGCGGAGCCTGCCGCTGCTGCTCAGCCAAGCTGGTGTGCGCACAGGCATC |
| | ATCGGGAAGAAGCACGTGGGGCCGGAGACCGTGTACCCGTTTGACTTTGCGTACACGGAG |
| | GAGAATGGCTCCGTCCTCCAGGTGGGGCGGAACATCACTAGAATTAAGCTGCTCGTCCGG |
| | AAATTCCTGCAGACTCAGGATGACCGGCCTTTCTTCCTCTACGTCGCCTTCCACGACCCC |
| | CACCGCTGTGGGCACTCCCAGCCCCAGTACGGAACCTTCTGTGAGAAGTTTGGCAACGGA |
| | GAGAGCGGCATGGGTCGTATCCCAGACTGGACCCCCCAGGCCTACGACCCACTGGACGTG |
| | CTGGTGCCTTACTTCGTCCCCAACACCCCGGCAGCCCGAGCCGACCTGGCCGCTCAGTAC |
| | ACCACCGTCGGCCGCATGGACCAAGGAGTTGGACTGGTGCTCCAGGAGCTGCGTGACGCC |
| | GGTGTCCTGAACGACACACTGGTGATCTTCACGTCCGACAACGGGATCCCCTTCCCCAGC |
| | GGCAGGACCAACCTGTACTGGCCGGGCACTGCTGAACCCTTACTGGTGTCATCCCCGGAG |
| | CACCCAAAACGCTGGGGCCAAGTCAGCGAGGCCTACGTGAGCCTCCTAGACCTCACGCCC |
| | ACCATCTTGGATTGGTTCTCGATCCCGTACCCCAGCTACGCCATCTTTGGCTCGAAGACC |
| | ATCCACCTCACTGGCCGGTCCCTCCTGCCGGCGCTGGAGGCCGAGCCCCTCTGGGCCACC |
| | GTCTTTGGCAGCCAGAGCCACCACGAGGTCACCATGTCCTACCCCATGCGCTCCGTGCAG |
| | CACCGGCACTTCCGCCTCGTGCACAACCTCAACTTCAAGATGCCCTTTCCCATCGACCAG |
| | GACTTCTACGTCTCACCCACCTTCCAGGACCTCCTGAACCGCACCACAGCTGGTCAGCCC |
| | ACGGGCTGGTACAAGGACCTCCGTCATTACTACTACCGGGCGCGCTGGGAGCTCTACGAC |
| | CGGAGCCGGGACCCCCACGAGACCCAGAACCTGGCCACCGACCCGCGCTTTGCTCAGCTT |
| | CTGGAGATGCTTCGGGACCAGCTGGCCAAGTGGCAGTGGGAGACCCACGACCCCTGGGTG |
| | TGCGCCCCCGACGGCGTCCTGGAGGAGAAGCTCTCTCCCCAGTGCCAGCCCCTCCACAAT |
| | GAGCTGTGA (SEQ ID NO: 44) |
| rAAV2 | Ctgcgcgctcgctcgctcactgaggccgcccgggcaaagcccgggcgtcgggcgaccttt |
| 5′ITR | ggtcgcccggcctcagtgagcgagcgagcgcgcagagagggagtggccaactccatcact |
| | aggggttcct (SEQ ID NO: 45) |
| 3x CRM8 | gggggaggctgctggtgaatattaaccaaggtcaccccagttatcggaggagcaaacagg |
| | ggctaagtccaccgggggaggctgctggtgaatattaaccaaggtcaccccagttatcgg |
| | aggagcaaacaggggctaagtccaccgggggaggctgctggtgaatattaaccaaggtca |
| | ccccagttatcggaggagcaaacaggggctaagtccac (SEQ ID NO: 46) |
| hTTR | aaatgacctattaagaatatttcatagaacgaatgttccgatgctctaatctctctagac |
| | aaggttcatatttgtatgggttacttattctctctttgttgactaagtcaataatcagaa |
| | tcagcaggtttgcagtcagattggcagggataagcagcctagctcaggagaagtgagtat |
| | aaaagccccaggctgggagcagccatcacagaagtccactcattcttggcagg (SEQ |
| | ID NO: 47) |
| MVM intron | ctaaggtaagttggcgccgtttaagggatggttggttggtggggtattaatgtttaatta |
| | ccttttttacaggcctg (SEQ ID NO: 48) |
| sv40PolyA | agatcttttaaaaaacctcccacacaattgttgttgttaacttgtttattgcagcttata |
| | atggttacaaataaagcaatagcatcacaaatttcacaaataaagcatttttttcactgc |
| | attctagttgtggtttgtccaaactcatcaatgtatcttatcatgtct (SEQ ID NO: |
| | 49) |
| rAAV2 | aggaacccctagtgatggagttggccactccctctctgcgcgctcgctcgctcactgagg |
| 3′ITR | ccgggcgaccaaaggtcgcccgacgcccgggctttgcccgggcggcctcagtgagcgagc |
| | gagcgcgcagaga (SEQ ID NO: 50) |
| 3XG4S | GGCGGTGGAGGCAGTGGTGGCGGGGGCTCCGGGGGTGGCGGTTCA (SEQ ID NO: |
| Linker | 51) |

In some aspects, the biologically active polypeptide has a glucocerebrosidase activity. In some aspects, the biologically active polypeptide is a glucocerebrosidase (GCB) or a functional variant thereof. In some aspects, the biologically active polypeptide comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 41, wherein the biologically active polypeptide has a glucocerebrosidase activity. In some aspects, the biologically active polypeptide comprises the amino acid sequence set forth in SEQ ID NO: 41.

In some aspects, the nucleotide sequence encoding the biologically active polypeptide comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 42, wherein the biologically active polypeptide encoded by the nucleotide sequence has a glucocerebrosidase activity. In some aspects, the nucleotide sequence encoding the biologically active polypeptide comprises the nucleic acid sequence set forth in SEQ ID NO: 42.

In some aspects, the biologically active polypeptide has an N-Sulfoglucosamine Sulfohydrolase (sulfamidase) activity. In some aspects, the biologically active polypeptide is an N-Sulfoglucosamine Sulfohydrolase (SGSH) or a functional variant thereof. In some aspects, the biologically active polypeptide comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 43, wherein the biologically active polypeptide has an N-Sulfoglucosamine Sulfohydrolase (sulfamidase) activity. In some aspects, the biologically active polypeptide comprises the amino acid sequence set forth in SEQ ID NO: 43.

In some aspects, the nucleotide sequence encoding the biologically active polypeptide comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 44, wherein the biologically active polypeptide encoded by the nucleotide sequence has an N-Sulfoglucosamine Sulfohydrolase (sulfamidase) activity. In some aspects, the nucleotide sequence encoding the biologically active polypeptide comprises the nucleic acid sequence set forth in SEQ ID NO: 44.

### B. TAG Moieties

In some aspects, the TAG increases the translocation of the biologically active polypeptide across the blood brain barrier. In some aspects, the TAG interacts with a receptor on the exterior surface of an endothelial cell of the blood brain barrier. In some aspects, the TAG comprises an antibody, an antigen-binding portion of an antibody, a ligand, or any combination thereof. In some aspects, the TAG comprises an antibody, wherein the antibody is a VHH, a vNAR, scFv, a diabody, a nanobody, a camelid antibody, an antigen-binding portion thereof, or any combination thereof. In some aspects, the TAG comprises a VHH. In some aspects, the TAG comprises a vNAR. In some aspects, the TAG comprises an scFv. In some aspects, the TAG comprises a nanobody.

In some aspects, the TAG interacts with a transferrin receptor on the exterior surface of an endothelial cell of the blood brain barrier. In some aspects, the TAG comprises an antigen-binding molecule that specifically binds a transferrin receptor on the exterior surface of an endothelial cell of the blood brain barrier. In some aspects, the TAG comprises an antigen-binding portion of an antibody that specifically binds a transferrin receptor on the exterior surface of an endothelial cell of the blood brain barrier. In some aspects, the TAG comprises a VHH that specifically binds a transferrin receptor on the exterior surface of an endothelial cell of the blood brain barrier. In some aspects, the VHH specifically binds human transferrin receptor 1 (TfR1).

In some aspects, the TAG comprises an antigen-binding molecule comprising a variable heavy (VH) domain, wherein the VH comprises a VH-CDR1, a VH-CDR2, and a VH-CDR3. In some aspects, the VH-CDR-3 comprises the amino acid sequence encoded by the nucleic acid sequence set forth in SEQ ID NO: 13. In some aspects, the VH-CDR-2 comprises the amino acid sequence encoded by the nucleic acid sequence set forth in SEQ ID NO: 12. In some aspects, the VH-CDR-1 comprises the amino acid sequence encoded by the nucleic acid sequence set forth in SEQ ID NO: 11. In some aspects, the TAG comprises a VH-CDR1 comprising the amino acid sequence encoded by the nucleic acid sequence set forth in SEQ ID NO: 11, a VH-CDR2 comprising the amino acid sequence encoded by the nucleic acid sequence set forth in SEQ ID NO: 12, and a VH-CDR3 comprising the amino acid sequence encoded by the nucleic acid sequence set forth in SEQ ID NO: 13, wherein the TAG specifically binds TfR1.

In some aspects, the TAG comprises a VHH comprising a VH-CDR1 comprising the amino acid sequence encoded by the nucleic acid sequence set forth in SEQ ID NO: 11, a VH-CDR2 comprising the amino acid sequence encoded by the nucleic acid sequence set forth in SEQ ID NO: 12, and a VH-CDR3 comprising the amino acid sequence encoded by the nucleic acid sequence set forth in SEQ ID NO: 13, wherein the VHH specifically binds TfR1.

In some aspects the VHH is encoded by a nucleic acid sequence having at least about 60%, at least about 65%, at least about 70%, at least about 75%, at least about 80%, at least about 85%, at least about 90%, at least about 95% at least about 96%, at least about 97% at least about 98%, or at least about 99% identity to the nucleic acid sequence set forth in SEQ ID NO: 14, 15, 16, 17, 18, 19, 20, or 21. In some aspects the VHH is encoded by a nucleic acid sequence set forth in SEQ ID NO: 14. In some aspects the VHH is encoded by a nucleic acid sequence set forth in SEQ ID NO: 15. In some aspects the VHH is encoded by a nucleic acid sequence set forth in SEQ ID NO: 16. In some aspects the VHH is encoded by a nucleic acid sequence set forth in SEQ ID NO: 17. In some aspects the VHH is encoded by a nucleic acid sequence set forth in SEQ ID NO: 18. In some aspects the VHH is encoded by a nucleic acid sequence set forth in SEQ ID NO: 19. In some aspects the VHH is encoded by a nucleic acid sequence set forth in SEQ ID NO: 20. In some aspects the VHH is encoded by a nucleic acid sequence set forth in SEQ ID NO: 21.

In some aspects, the TAG is any anti-TfR binding protein disclosed in International Publication No. WO/2020/144233, which is incorporated by reference herein in its entirety.

TABLE 2
TAG Sequences

| | |
|---|---|
| VHH-CDR1 | GGAACGGACTTCAGTATCAATTTT (SEQ ID NO: 11) |
| VHH-CDR2 | TTTACTGCGACTGGTAACACA (SEQ ID NO: 12) |
| VHH-CDR3 | TATATGTTGGACAAG (SEQ ID NO: 13) |
| VHH-1 | GAGGTGCAGCTGGTGGAGTCTGGGGGAGGCGTGGTGCAGCCTGGGGGGTCTCTAAAAC |
| | TCTCCTGCGTAGCCTCGGGAACGGACTTCAGTATCAATTTTATACGCTGGTACCGCCA |
| | GGCTCCAGGGAAGCAGCGCGAGTTCGTCGCAGGATTTACTGCGACTGGTAACACAAAC |
| | TATGCAGACTCCATGAAGGGGCGATTCACCATCTCCAGAGACAACACCAAGAACGCGG |
| | TGTATCTGCAAATAGACAGCCTGAAACCTGAGGACACGGCCGTGTATTACTGCTATAT |
| | GTTGGACAAGTGGGGCCAGGGGACCCAGGTCACCGTCTCCTCA (SEQ ID NO: |
| | 14) |
| VHH-2 | GAGGTGCAGCTGGTGGAGTCTGGGGGAGGCGTGGTGCAGCCTGGGGGGTCTCTAAAAC |
| | TCTCCTGCGTAGCCTCGGGAACGGACTTCAGTATCAATTTTATACGCTGGTACCGCCA |
| | GGCTCCAGGGAAGCAGCGCGAGTTCGTCGCAGGATTTACTGCGACTGGTAACACAAAC |
| | TATGCAGACTCCATGAAGGGGCGATTCACCATCTCCAGAGACAACACCAAGAACGCGG |
| | TGTATCTGCAAATAGACAGCCTGAAACCTGAGGACACGGCCGTGTATTACTGCTATAT |
| | GATTGACAAGTGGGGCCAGGGGACCCAGGTCACCGTCTCCTCA (SEQ ID NO: |
| | 15) |
| VHH-3 | GAGGTGCAGCTGGTGGAGTCTGGGGGAGGCGTGGTGCAGCCTGGGGGGTCTCTACGTC |
| | TCTCCTGCGCAGCCTCGGGAACGGACTTCAGTATCAATTTTATGAGCTGGGTTCGCCA |
| | GGCTCCAGGGAAGGGTCTGGAGTGGGTCGCAGGATTTACTGCGACTGGTAACACAAAC |
| | TATGCAGACTCCGTTAAGGGGCGATTCACCATCTCCAGAGACAACGCAAAGAACACCC |
| | TGTATCTGCAAATGAATAGCCTGCGTCCTGAGGACACGGCCGTGTATTACTGCTATAT |
| | GTTGGACAAGTGGGGCCAGGGGACCCAGGTCACCGTCTCCTCA (SEQ ID NO: |
| | 16) |
| VHH-4 | GAGGTGCAGCTGGTGGAGTCTGGGGGAGGCGTGGTGCAGCCTGGGGGGTCTCTACGTC |
| | TCTCCTGCGCAGCCTCGGGAACGGACTTCAGTATCAATTTTATACGCTGGGTTCGCCA |
| | GGCTCCAGGGAAGCAGCGCGAGTTCGTCGCAGGATTTACTGCGACTGGTAACACAAAC |
| | TATGCAGACTCCGTTAAGGGGCGATTCACCATCTCCAGAGACAACGCAAAGAACACCC |
| | TGTATCTGCAAATGAATAGCCTGCGTCCTGAGGACACGGCCGTGTATTACTGCTATAT |
| | GTTGGACAAGTGGGGCCAGGGGACCCAGGTCACCGTCTCCTCA (SEQ ID NO: |
| | 17) |
| VHH-5 | GAGGTGCAGCTGGTGGAGTCTGGGGGAGGCGTGGTGCAGCCTGGGGGGTCTCTACGTC |
| | TCTCCTGCGCAGCCTCGGGAACGGACTTCAGTATCAATTTTATGCATTGGGTTCGCCA |
| | GGCTCCAGGGAAGGGTCTGGAGTGGGTCGCAGGATTTACTGCGACTGGTAACACAAAC |
| | TATGCAGACTCCGTTAAGGGGCGATTCACCATCTCCAGAGACAACGCAAAGAACACCC |
| | TGTATCTGCAAATGAATAGCCTGCGTCCTGAGGACACGGCCGTGTATTACTGCTATAT |
| | GTTGGACAAGTGGGGCCAGGGGACCCAGGTCACCGTCTCCTCA (SEQ ID NO: |
| | 18) |
| VHH-6 | GAGGTGCAGCTGGTGGAGTCTGGGGGAGGCGTGGTGCAGCCTGGGGGGTCTCTACGTC |
| | TCTCCTGCGCAGCCTCGGGAACGGACTTCAGTATCAATTTTATGAGCTGGGTTCGCCA |
| | GGCTCCAGGGAAGCAGCGCGAGTTCGTCGCAGGATTTACTGCGACTGGTAACACAAAC |
| | TATGCAGACTCCGTTAAGGGGCGATTCACCATCTCCAGAGACAACGCAAAGAACACCC |
| | TGTATCTGCAAATGAATAGCCTGCGTCCTGAGGACACGGCCGTGTATTACTGCTATAT |
| | GTTGGACAAGTGGGGCCAGGGGACCCAGGTCACCGTCTCCTCA (SEQ ID NO: |
| | 19) |
| VHH-7 | GAGGTGCAGCTGGTGGAGTCTGGGGGAGGCGTGGTGCAGCCTGGGGGGTCTCTACGTC |
| | TCTCCTGCGCAGCCTCGGGAACGGACTTCAGTATCAATTTTATACGCTGGGTTCGCCA |
| | GGCTCCAGGGAAGGGTCTGGAGTGGGTCGCAGGATTTACTGCGACTGGTAACACAAAC |
| | TATGCAGACTCCGTTAAGGGGCGATTCACCATCTCCAGAGACAACGCAAAGAACACCC |
| | TGTATCTGCAAATGAATAGCCTGCGTCCTGAGGACACGGCCGTGTATTACTGCTATAT |
| | GTTGGACAAGTGGGGCCAGGGGACCCAGGTCACCGTCTCCTCA (SEQ ID NO: |
| | 20) |
| VHH-8 | GAGGTGCAGCTGGTGGAGTCTGGGGGAGGCGTGGTGCAGCCTGGGGGGTCTCTACGTC |
| | TCTCCTGCGCAGCCTCGGGAACGGACTTCAGTATCAATTTTATACATTGGGTTCGCCA |
| | GGCTCCAGGGAAGGGTCTGGAGTGGGTCGCAGGATTTACTGCGACTGGTAACACAAAC |
| | TATGCAGACTCCGTTAAGGGGCGATTCACCATCTCCAGAGACAACGCAAAGAACACCC |
| | TGTATCTGCAAATGAATAGCCTGCGTCCTGAGGACACGGCCGTGTATTACTGCTATAT |
| | GTTGGACAAGTGGGGCCAGGGGACCCAGGTCACCGTCTCCTCA |
| | (SEQ ID NO: 21) |

### C. Viral Vectors

Transgenes delivered by the vector can be introduced into a cell of interest using a variety of methods. For example, either viral or non-viral vectors can be used for the delivery of a transgene of interest. Both viral and non-viral methods of vector delivery are contemplated by the methods provided herein. Accordingly, in some aspects, the vector described herein is delivered in a viral vector. In some aspects, the vector described herein is delivered in a non-viral vector.

A vector as described herein can be introduced into a cell as a part of a viral or non-viral vector molecule having additional sequences, such as, for example, replication origins, promoter and one or more genes. In some aspects, the vectors can be introduced as naked nucleic acids, as nucleic acid complexed with an agent such as a liposome or a poloxamer, or can be delivered by viruses (e.g., adenovirus, adeno-associated virus (AAV), herpesvirus, retrovirus, lentivirus and integrase defective lentivirus (IDLV). In some aspects, the vector is introduced using a viral vector.

Various viral vectors are known in the art, and include for example either integrating or non-integrating vectors. In some aspects, the viral vector is a non-integrating viral vector. Non-integrating viral vectors include, for example non-integrating lentivirus vectors and AAV vectors. Accordingly, in some aspects, the viral vector is an adeno-associated virus (AAV) vector.

In some aspects, the AAV vector is modified at one or more regions, such as the AAV capsid. In some aspects, the viral vector is selected from one or more of AAV1, AAV2, AAV3, AAV4, AAV5, AAV6, AAV7, AAV8 or AAV9 having a ubiquitous promoter. In some aspects, an appropriate viral vector with wide tropism can be engineered with combined elements of AAV1, AAV2, AAV3, AAV4, AAV5, AAV6, AAV7, AAV8 or AAV9 having a ubiquitous promoter.

In some aspects, a viral vector encompassed by the present disclosure comprises a tissue specific promoter, e.g., a liver specific promoter upstream of a nucleic acid sequence encoding an I2S polypeptide linked to an anti-TfR1 VHH.

In some aspects, an intrinsic transgene expression system as provided herein comprises a viral vector that improves the exposure or distribution of the transgene, e.g., encoding an I2S polypeptide linked to an anti-TfR1 VHH, in various tissues in a mammal. In some aspects, the improved exposure or distribution of I2S polypeptide linked to an anti-TfR1 VHH in various tissues improves the symptoms associated with, e.g., Hunter syndrome. In some aspects, the use of a viral vector complements the use of ubiquitous promoter in providing robust tissue distribution of the I2S polypeptide linked to an anti-TfR1 VHH. In some aspects, the viral vector is selected from one or more of AAV1, AAV2, AAV3, AAV4, AAV5, AAV6, AAV7, AAV8 or AAV9 having a ubiquitous promoter. In some aspects, an appropriate viral vector with wide tropism can be engineered with combined elements of AAV1, AAV2, AAV3, AAV4, AAV5, AAV6, AAV7, AAV8 or AAV9 having a ubiquitous promoter. In some aspects, the rAAV vector is a rAAV9 vector.

In some aspects, the rAAV vector described herein comprises one or more of: (a) a 5′ inverted terminal repeat (ITR); (b) a promoter sequence; (c) a transgene encompassed by the disclosure herein comprising a nucleic acid sequence encoding a biologically active polypeptide and a nucleic acid encoding a tag; (d) a woodchuck hepatitis virus posttranscriptional regulatory element (WPRE), (e) a poly A; and (f) a 3′ ITR sequence. In some aspects, the rAAV vector described herein comprises one or more of: (a) a 5′ inverted terminal repeat (ITR); (b) an enhancer sequence (c) a promoter sequence; (d) a transgene encompassed by the disclosure herein; (e) a woodchuck hepatitis virus posttranscriptional regulatory element (WPRE), (f) a poly A; and (g) a 3′ ITR sequence. In some embodiments, the transgene further comprises a nucleic acid linker sequence between the nucleic acid encoding the polypeptide and the nucleic acid sequence encoding the tag.

In various aspects, a rAAV vector described herein for delivering a transgene encompassed by the disclosure herein can be packaged using techniques known in the art and as described herein. For example, in some aspects, rAAV packaging makes use of packaging cells to form virus particles that are capable of infecting a host cell. Such cells include, for example HEK293, HeLa, HEK293T, Sf9 cells or A549 cells, which are used to package adenovirus. Viral vectors used in gene therapy are usually generated by a producer cell line that packages a nucleic acid vector into a viral particle. The vectors typically contain the minimal viral sequences required for packaging nucleic acid and subsequent transduction of a host, other viral sequences being replaced by an expression cassette encoding the protein to be expressed. In this case the transgene comprising the nucleotide sequence encoding the biologically active polypeptide and the nucleotide sequence encoding the TAG. The missing viral functions can be supplied in trans by the packaging cell line. For example, AAV vectors used in gene therapy typically only possess inverted terminal repeat (ITR) sequences from the AAV genome which are required for packaging and transduction into the host cell. Viral DNA is packaged in a cell line, which contains a helper plasmid encoding the other AAV genes, namely rep and cap, but lacking ITR sequences. The cell line is also transfected with adenovirus plasmid as a helper. The helper plasmid promotes replication of the AAV vector and encapsidation of the nucleic acid into proteinaceous capsid. The helper plasmid is not packaged into the AAV due to a lack of ITR sequences and packaging size constraints. Contamination with adenovirus or adenovirus-derived plasmid can be reduced by inactivation during purification, e.g., heat treatment to which adenovirus is more sensitive than AAV.

In many gene therapy applications, it is desirable that the gene therapy vector be delivered with a specificity to a particular tissue type. As such, the vector design provided here has a wide tissue and cell type distribution once administered to a subject in need thereof. The rAAV vectors described herein can comprise any tissue-specific or constitutively active promoter, allowing for systemic expression or specific expression in a particular tissue, such as the liver.

In some aspects, the present disclosure encompasses a gene therapy vector comprising a biologically active polypeptide-encoding gene, e.g., an I2S gene, sequence that is modified. Such modification may be made to improve expression characteristics. Such modifications can include, but are not limited to, insertion of a translation start site (e.g. methionine), addition of a Kozak sequence, insertion of a signal peptide, and/or codon optimization. Accordingly, in some aspects, the biologically active polypeptide-encoding gene, e.g., an I2S gene, is modified to include insertion of a translation start site. In some aspects, the biologically active polypeptide-encoding gene, e.g., an I2S gene, is modified to include the addition of a Kozak sequence. In some aspects, the biologically active polypeptide-encoding gene, e.g., an I2S gene, is modified to comprise a signal peptide. In some aspects, the signal peptide comprises an immunoglobulin signal peptide. In some aspects, the signal peptide comprises an IgG signal peptide. In some aspects, the biologically active polypeptide-encoding gene, e.g., an I2S gene, is codon optimized. In other aspects, the biologically active polypeptide-encoding gene, e.g., an I2S gene, is engineered. In yet other aspects, the biologically active polypeptide-encoding gene, e.g., an I2S gene, is codon optimized and engineered.

In some aspects, the vector comprises an ID tag, e.g., a stuffer sequence. The purpose of the ID tag includes for example the ability for an artisan to identify the vector. In certain aspects, the vector comprises woodchuck hepatitis virus posttranscriptional regulatory element (WPRE) element. In some aspects, the vector comprises woodchuck hepatitis virus post-transcriptional control element (WPRE). Various optimized or variant forms of WPRE are known in the art, and include WPRE3, WPREmut6delATG among others. Other variant WPRE forms include, for example, WPRE2, WPRE_wt (GenBank accession no. J04514); WPRE_wt (GenBank accession no. J02442) and WPREmut6. The WPRE element can comprises a wild-type sequence or a modified WPRE element sequence. Various mutated versions of WPRE are known, and include for example, mut6delATG. In some aspects, the vector comprises mut6delATG.

The vector described herein comprises one or more promoter sequences. In some aspects, the promoter sequence is a ubiquitous promoter sequence. Any suitable promoter region or promoter sequence can be used, so long as the promoter region promotes expression of a coding sequence in mammalian cells. In certain aspects, the promoter region promotes expression of a coding sequence in mammalian cells. In some aspects, the promoter controlling the expression of the transgene is a ubiquitous promoter. In some aspects, the ubiquitous promoter is selected from one or more of GAPDH promoter, mini EF1 promoter, CMV promoter EF-1α promoter, PGK promoter, UBC promoter, LSE beta-glucuronidase (GUSB) promoter, or ubiquitous chromatin opening element (UCOE) and/or chicken beta actin promoter. In some aspects, the ubiquitous promoter comprises ubiquitous promoter comprising a cyto-megalo-virus (CMV) enhancer, chicken beta actin promoter (CBA), and a rabbit beta globin intron.

In some aspects, the promoter sequence is a tissue-specific promoter sequence. In certain aspects, the promoter region promotes expression of a coding sequence in the liver, i.e., a liver-specific promoter. In some aspects, the promoter comprises a human TTR, PGK, chicken β actin (CBA) promoter, CAG promoter, EF-1α promoter, UBC promoter, LSE beta-glucuronidase (GUSB) promoter, or ubiquitous chromatin opening element (UCOE) promoter.

In some embodiments, the ubiquitous promoter comprises CBh (CMV enhancer, Chicken beta-actin promoter, Chicken-beta actin-MVM hybrid intron). Accordingly, in some embodiments, the ubiquitous promoter is a chicken β actin (CBA) promoter. In some embodiments, the ubiquitous promoter is an EF-1α promoter. In some embodiments, the EF-1α promoter is in combination with chimeric intron from chicken β-actin and rabbit β-globin genes. In some embodiments, the ubiquitous promoter is a UBC promoter. In some embodiments, the ubiquitous promoter is an LSE beta-glucuronidase (GUSB) promoter. In some embodiments, the ubiquitous promoter is a ubiquitous chromatin opening element (UCOE) promoter. (Powell S K, et al. Discov Med. 2015 January; 19(102):49-57.)

In some embodiments, the ubiquitous promoter comprises a cyto-megalo-virus (CMV) enhancer, chicken beta actin promoter, and a rabbit beta globin intron.

In some embodiments, the ubiquitous promoter comprises a shortened EF-1α promoter and one or more introns.

In some embodiments, the one or more introns are from chicken β-actin and/or rabbit β-globin genes.

In some aspects, the promoter comprises the nucleic acid sequence:

(SEQ ID NO: 30)
ggggttggggttgcgccttttccaaggcagccctgggtttgcgcagg
gacgcggctgctctgggcgtggttccgggaaacgcagcggtgccgac
cctgggtctcgcacattcttcacgtccgttcgcagcgtcacccggat
cttcgccgctacccttgtgggccccccggcgacgcttcctgctccgc
ccctaagtcgggaaggttccttgcggttcgcggcgtgccggacgtga
caaacggaagccgcacgtctcactagtaccctcgcagacggacagcg
ccagggagcaatggcagcgcgccgaccgcgatgggctgtggccaata
gcggctgctcagcggggcgcgccgagagcagcggccgggaaggggcg
gtgcgggaggcggggtgtggggcggtagtgtgggccctgttcctgcc
cgcgcggtgttccgcattctgcaagcctccggagcgcacgtcggcag
tcggctccctcgttgaccgaatcaccgacctctctccccag

In some aspects, the promoter comprises the nucleic acid sequence:

(SEQ ID NO: 31)
aaatgacctattaagaatatttcatagaacgaatgttccgatgctct
aatctctctagacaaggttcatatttgtatgggttacttattctctc
tttgttgactaagtcaataatcagaatcagcaggtttgcagtcagat
tggcagggataagcagcctagctcaggagaagtgagtataaaagccc
caggctgggagcagccatcacagaagtccactcattcttggcagg

In some aspects, the vector described herein comprises one or more polyA sequences. In some aspects, the polyA is selected from human growth hormone polyA (hGHpA), synthetic polyA (SPA), Simian virus 40 late poly A (SV40pA) and a bovine growth hormone (BGH) poly A.

In some aspects, the disclosure provides an expression cassette comprising a polynucleotide sequence comprising: (a) a 5′ inverted terminal repeat (ITR); (b) a AAT enhancer and an hTTR promoter; (c) a transgene comprising (i) a nucleotide sequence encoding I2S and a linker polypeptide and (ii) a nucleotide sequence encoding a VHH that specifically binds hTfR1; (d) optionally a woodchuck hepatitis virus posttranscriptional regulatory element (WPRE) comprising the mut6delATG mutation; (e) a poly A; and (f) a 3′ ITR. In some aspects, the elements in the expression cassette above are present in 5′ to 3′ order. In some aspects, one or more of (a) to (f) are operably linked in 5′ to 3′ order.

In some aspects, the vector is introduced into a cell. Accordingly, in some aspects, a cell is provided, said cell comprising a vector described herein. In some aspects, a cell is in vitro, in situ, or in vivo. Accordingly, in some aspects, the cell comprising the vector described herein is in vitro. In some aspects, the cell comprising the vector described herein is in situ. In some aspects, the cell comprising the vector described herein is in vivo.

### D. Pharmaceutical Compositions

Exemplary pharmaceutical compositions comprising the vectors described herein are detailed below.

Pharmaceutically acceptable carriers are determined in part by the particular composition being administered, as well as by the particular method used to administer the compositions. Accordingly, there is a wide variety of suitable formulations of pharmaceutical compositions available.

Formulations for both ex vivo and in vivo administrations include suspensions in liquid or emulsified liquids. The active ingredients often are mixed with excipients that are pharmaceutically acceptable and compatible with the active ingredient. Suitable excipients include, for example, water, saline, dextrose, glycerol, ethanol, or the like, and combinations thereof. In addition, the compositions may contain minor amounts of auxiliary substances, such as wetting or emulsifying agents, pH buffering agents, stabilizing agents or other reagents that enhance the effectiveness of the pharmaceutical composition.

### III. Methods of the Disclosure

Some aspects of the present disclosure are directed to methods of treating a disease or condition in a subject in need thereof comprising administering to the subject a viral vector, e.g., a rAAV vector, disclosed herein. Some aspects of the present disclosure are directed to methods of delivering a biologically active polypeptide across the blood brain barrier in a subject, comprising administering to the subject a viral vector, e.g., a rAAV vector, disclosed herein.

In some aspects, the disease or condition comprises a neuronal disease. In some aspects, the disease or condition comprises one or more presentation that affects the CNS. In some aspects, the disease or condition comprises a deficiency in the CNS. In some aspects, the disease or condition comprises a deficit in the CNS.

In some aspects, the disease or conditions comprises accumulation of a high concentration of a substrate in the CNS of the subject. In some aspects, the disease or conditions comprises accumulation of a high concentration of I2S substrate (e.g., lysosomal glycosaminoglycans (GAGs)) in the CNS of the subject. In some aspects, the disease or condition comprises a mucopolyscahharidoses (MPS). In some aspects, the disease or condition comprises Hurler syndrome (also known as MPS I). In some aspects, the disease or condition comprises Hunter syndrome (also known as MPSII). In some aspects, the disease or condition comprises Sanfilippo syndrome (also known as MPS III). In some aspects, the disease or condition comprises Sly syndrome (also known as MPS VII). In some aspects, the disease or condition comprises Gaucher disease. In some aspects, the disease or condition comprises Metachromatic leukodystrophy. In some aspects, the disease or condition comprises Krabbe disorder. In some aspects, the disease or condition comprises GM1 gangliosidosis.

In some aspects, the disease or condition comprises an I2S deficiency (e.g., Hunter syndrome). In some aspects, administration of the viral vector, e.g., rAAV vector, disclosed herein results in increased levels of I2S in the subject. In some aspects, increased I2S levels are observed systemically. In some aspects, administration of the viral vector, e.g., rAAV vector, disclosed herein results in increased levels of I2S in the CNS of the subject. In some aspects, the increased I2S levels persist for at least about 1 week, at least about 2 weeks, at least about 3 weeks, at least about 4 weeks, at least about 6 weeks, at least about 8 weeks, at least about 10 weeks, at least about 3 months, at least about 4 months, at least about 5 months, at least about 6 months, at least about 9 months, at least about 12 months, at least about 15 months, at least about 18 months, at least about 21 months, at least about 2 years, or longer. In some aspects, functional levels of the I2S polypeptide is detectable in plasma, serum, of CSF for at least about 1 year, at least about 2 years, at least about 3 years, at least about 4 years, at least about 5 years, at least about 6 years, at least about 7 years, at least about 8 years, at least about 9 years, at least about 10 years, at least about 15 years, or at least about 20 years after administration of the viral vector, e.g., rAAV vector.

In some aspects, administration of the viral vector, e.g., rAAV vector, disclosed herein results in decreased levels of GAGs in the subject. In some aspects, GAGs levels are reduced systemically and within the CNS. In some aspects, GAGs levels following administration of the viral vector, e.g., AAV vector, disclosed herein are less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 4%, less than about 3%, less than about 2%, or less than about 1%, less than about as compared to the level of the GAGs in the subject prior to the administration. In some aspects, the level of GAGs in the subject following administration of the viral vector, e.g., AAV vector, is comparable to the level of the GAGs in a healthy subject, e.g., ±less than about 10%, ±less than about 9%, ±less than about 8%, ±less than about 7%, ±less than about 6%, less than about 5%, ±less than about 4%, ±less than about 3%, ±less than about 2%, less than about %, or ±less than about 1%.

In some aspects, GAGs levels in the brain of the subject following administration of the viral vector, e.g., AAV vector, disclosed herein are less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 4%, less than about 3%, less than about 2%, or less than about 1%, less than about as compared to the level of the I2S substrate in the brain of the subject prior to the administration. In some aspects, the level of GAGs in the brain of the subject following administration of the viral vector, e.g., AAV vector, is comparable to the level of GAGs in the brain of a healthy subject, e.g., ±less than about 10%, ±less than about 9%, less than about 8%, ±less than about 7%, less than about 6%, ±less than about 5%, less than about 4%, ±less than about 3%, less than about 2%, ±less than about %, or less than about 1%.

In some aspects, the administered rAAV comprising the I2S transgene reduces the level of GAGs in the subject for at least about 2 weeks, 1 month, 2 months, 3 months, 4 months, 5 months, 6 months, 12 months, 1 year, 2 years, 3 years, 4 years, 5 years, or more than 5 years. In some aspects, the administered rAAV comprising the I2S transgene reduces the level of GAGs in the brain of the subject for at least about 2 weeks, 1 month, 2 months, 3 months, 4 months, 5 months, 6 months, 12 months, 1 year, 2 years, 3 years, 4 years, 5 years, or more than 5 years.

The viral vectors, e.g., rAAV vectors, disclosed herein can be administered using any route. In some aspects, the viral vector, e.g., rAAV vector, is administered by intravenous, intraperitoneal, intra-arterial, intrathecal (cisterna magna, lumbar puncture), subcutaneous, or intradermal administration. In some aspects, the viral vector, e.g., rAAV vector, is administered intravenously. In some aspects, the intradermal administration comprises administration by use of a “gene gun” or biolistic particle delivery system. In some aspects, the viral vector, e.g., rAAV vector, is administered via a non-viral lipid nanoparticle. For example, a composition comprising the viral vector, e.g., rAAV vector, can comprise one or more diluents, buffers, liposomes, a lipid, a lipid complex. In some aspects, the viral vector, e.g., rAAV vector, is comprised within a microsphere or a nanoparticle, such as a lipid nanoparticle.

The compositions and methods of the invention can also be used in conjunction with other remedies known in the art that are used to treat Hunter syndrome or its complications, including but not limited to Enzyme Replacement Therapy (e.g., I2S).

Methods for generating and isolating AAV viral vectors suitable for delivery to a subject are known in the art. See, e.g., U.S. Pat. Nos. 7,790,449; 7,282,199; WO 2003/042397; WO 2005/033321; WO 2006/110689; and U.S. Pat. No. 7,588,772 B2. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

In some aspects, the expression cassette flanked by ITRs and rep/cap genes are introduced into a desired cell or cell line by infection with baculovirus-based vectors.

In some aspects, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin Biotechnol. 99: 119-145; Buning et al., 2008, “Recent developments in adeno-associated virus vector technology,” J. Gene Med 10:717-733; and the references cited below, each of which is incorporated herein by reference in its entirety.

The methods used to construct a vector as described herein are known to those with skill in nucleic acid manipulation and include genetic engineering, recombinant engineering, and synthetic techniques. See, e.g., Green and Sambrook et al., Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al., (1993) J. Virol, 70:520-532 and U.S. Pat. No. 5,478,745.

Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In one aspect, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

In certain aspects, the rAAV expression cassette, the vector (such as rAAV vector), the virus (such as rAAV), the production plasmid comprises AAV inverted terminal repeat sequences, a codon optimized nucleic acid sequence that encodes an I2S polypeptide linked to an anti-TfR1 VHH sequence, and expression control sequences that direct expression of the encoded proteins are present in a host cell. In other aspects, the rAAV expression cassette, the virus, the vector (such as rAAV vector), the production plasmid further comprise one or more of an enhancer, promoter, intron, a Kozak sequence, a polyA, posttranscriptional regulatory elements and others. In one aspect, the post-transcriptional regulatory element is Woodchuck Hepatitis Virus (WHP) Posttranscriptional Regulatory Element (WPRE). In various aspects, the nucleic acid sequence comprises a signal peptide upstream of the transgene that encodes an I2S polypeptide linked to an anti-TfR1 VHH sequence. In some aspects, a signal peptide is at the N-terminus of an I2S polypeptide linked to an anti-TfR1 VHH sequence. In some aspects, a signal peptide is at the C-terminus of an I2S polypeptide linked to an anti-TfR1 VHH sequence.

Various methods are known in the art relating to the production and purification of AAV vectors. See, e.g., Mizukami, Hiroaki, et al. A Protocol for AAV vector production and purification, available at dnaconda.riken.jp/rvd/SOP/AAV/AAVProtocol.pdf; U.S. Patent Publication Numbers US20070015238 and US20120322861. For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV can be packaged and subsequently purified.

In some aspects, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprise rep and/or cap genes that encode the Rep protein and/or Cap proteins for use in a method described herein. In some aspects, the packaging is performed in vitro.

In some aspects, a plasmid containing comprising the gene of interest is combined with one or more helper plasmids, e.g., that contain a rep gene of a first serotype and a cap gene of the same serotype or a different serotype, and transfected into helper cells such that the rAAV is packaged.

In some aspects, the one or more helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA. In some aspects, the cap gene is modified such that one or more of the proteins VP1, VP2 and VP3 do not get expressed. In some aspects, the cap gene is modified such that VP2 does not get expressed. Methods for making such modifications are known in the art (Lux et al. (2005), J. Virology, 79: 11776-87).

Helper plasmids, and methods of making such plasmids, are generally known in the art and generally commercially available (see, e.g., pDF6, pRep, pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adeno associated Virus Vectors, Human Gene Therapy, Vol. 9, 2745-2760; Kem, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al., J. Virol. 79(9):5296-5303 (2005)).

### EXAMPLES

### Example 1—Assessment of Gene Therapy Constructs Expressing Either Liver Specific or Ubiquitous Promoter Driven VHH-I2S in a Mouse Model of Hunter Disease

Hunter disease is characterized by deficient activity of lysosomal enzyme iduronate-2-sulfatase (I2S). The deficient enzyme activity results in pathological accumulation of lysosomal glycosaminoglycans (GAGs), which ultimately results in progressive damage and dysfunction in cells, tissues and organs throughout the body.

This example shows that gene therapy constructs expressing either liver specific or ubiquitous promoter driven VHH-I2S achieve significantly higher brain activity and GAG clearance in a mouse model of Hunter disease than constructs expressing untagged I2S.

Presence of the VHH Tag does not Hamper I2S Expression or Catalytic Activity

Plasmids expressing I2S with and without the VHH tag (Table 3) were first transfected into Huh7 cells before being tested in a mouse model of Hunter disease. The molecular weight of the protein product of these plasmids confirms intactness of the tagged I2S. Purified protein was then analyzed for I2S activity. Notably, the activity of the tagged I2S proteins were similar to that of untagged I2S (Table 4). These results show that presence of the VHH tag does not hamper I2S catalytic activity.

Assessment of binding affinities of VHH-I2S and I2S-VHH to mouse and human TfR1 receptors were made via surface plasmon resonance analyses (Table 5). VHH-I2S binds to hTfR1 with sub-nanomolar affinity and to mouse TfR1 with single digit nanomolar affinity.

TABLE 3
A description of tool molecules with VHH fused to I2S driven
by a liver specific promoter (GTH077, GTH071, GTH074)
and a ubiquitous promoter (GTH075, GTH069, GTH072)

| Constructs | Promoter | Transgene |
|---|---|---|
| GTH077 | Liver specific | I2S |
| GTH071 | Liver specific | I2S-VHH |
| GTH074 | Liver specific | VHH-I2S |
| GTH075 | Ubiquitous | I2S |
| GTH069 | Ubiquitous | I2S-VHH |
| GTH072 | Ubiquitous | VHH-I2S |

TABLE 4
hI2S activity is not impacted by VHH tag

| | I2S activity (nmol 4MU/hr/nmol protein) |
|---|---|
| Purified protein | AVG ± standard deviation |
| I2S control | 101 ± 36 |
| I2S-VHH | 106 ± 27 |
| VHH-I2S | 87 ± 33 |

TABLE 5
Preliminary binding affinity of purified hI2S proteins
to mouse and human transferrin receptor 1 (TFR1)

| Protein | K_{d} mTFR1 (M) | K_{d} hTFR1 (M) |
|---|---|---|
| I2S control | no binding | no binding |
| I2S-VHH | Not quantified | 2.3E−08 |
| VHH-I2S | 1.3E−09 | 1.6E−10 |

Liver Directed Gene Therapy Constructs Expressing VHH-I2S Show Significantly Higher Brain Activity and GAG Clearance than Constructs Expressing Untagged I2S.

Next, the plasmids expressing tagged and untagged I2S were packaged into rAAV9 capsid and evaluated in 8-12-weeks-old male IdsKO mice. rAAV9-MY011 (null) treated and wild-type (WT:WT) littermate vehicle treated groups were used as controls, and were evaluated in a 4-week study using a single intravenous administration at 2.5×10¹² vg/kg and 6.25×10¹² vg/kg dose for tool molecules with a liver-specific promoter (GTH077, GTH071, GTH074) and only at 2.5×10¹² vg/kg for tool molecules with a ubiquitous promoter (GTH075, GTH069, GTH072).

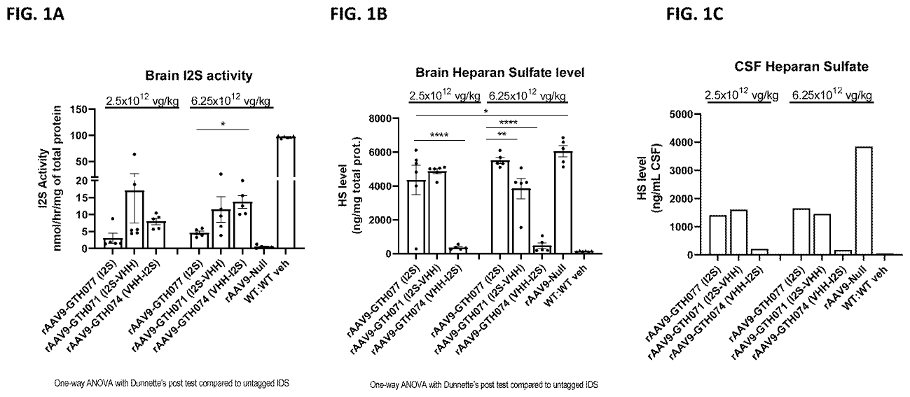

Mice treated with rAAV9-GTH071 (I2S-VHH) and rAAV9-GTH074 (VHH-I2S) showed significantly higher brain hI2S activity compared to mice treated with untagged control rAAV9-GTH077 (I2S) (FIG. 1A). This resulted in a substantial reduction (>90%) of heparan sulfate (HS) GAGs in the brain (FIG. 1B and Table 6) and normalized CSF GAGs to WT levels (FIG. 1C and Table 6).

TABLE 6
Percentage Brain and CSF heparan sulfate reduction
in mice treated with rAAV9-GTH077 (I2S),
rAAV9-GTH071 (I2S-VHH) or rAAV9-GTH074
(VHH-I2S) at 2.5 × 10¹² vg/kg and
6.25 × 10¹² vg/kg
Percent Heparan Sulfate Reduction from I2SKO

| | Brain | | CSF | |
|---|---|---|---|---|
| Tool vector | 2.5 × 10¹² | 6.25 × 10¹² | 2.5 × 10¹² | 6.25 × 10¹² |
| molecules | vg/kg | vg/kg | vg/kg | vg/kg |
| rAAV9-GTH077 | 15% | 9% | 63% | 57% |
| (I2S) | | | | |
| rAAV9-GTH071 | 19% | 36% | 58% | 62% |
| (I2S-VHH) | | | | |
| rAAV9-GTH074 | 94% | 92% | 95% | 95% |
| (VHH-I2S) | | | | |

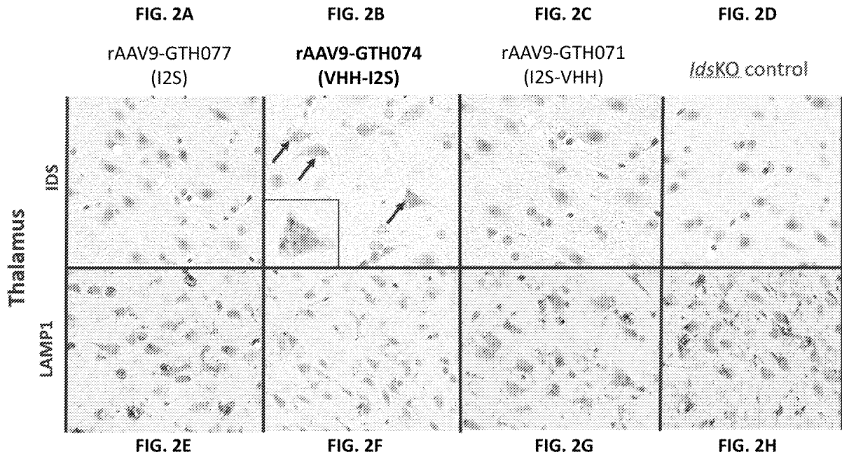

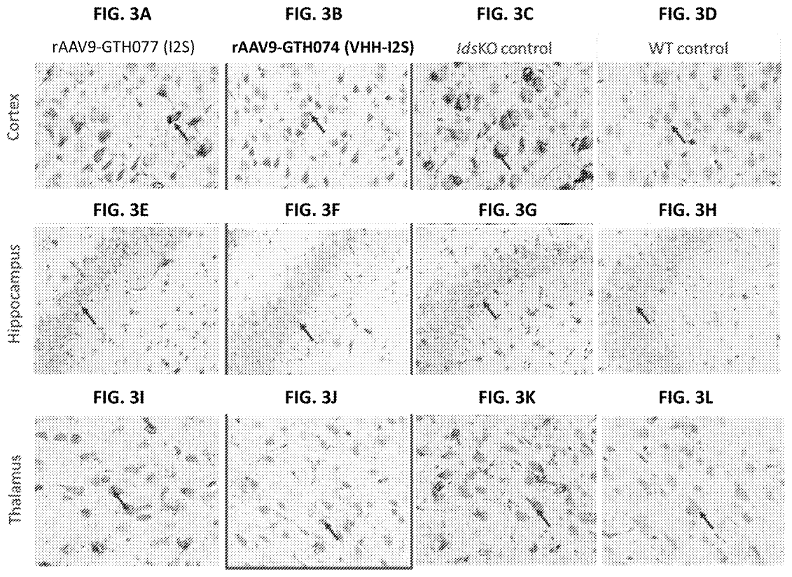

Immunohistochemical analyses of brain tissue confirmed hI2S exposure in the neurons in deeper brain regions such as the thalamus and hippocampus and subsequent reduction in lysosomal burden as measured by LAMP1 staining (FIGS. 2 and 3). Specifically, intravenous administration of rAAV9-GTH074 (VHH-I2S) normalized LAMP1 staining to WT levels in the cortex, hippocampus, and thalamus of I2SKO mice. This clearly demonstrated that the N terminally tagged VHH-I2S protein produced in liver was able to penetrate across the BBB more efficiently to cross-correct HS GAG accumulation in the brain and CSF significantly over the untagged I2S.

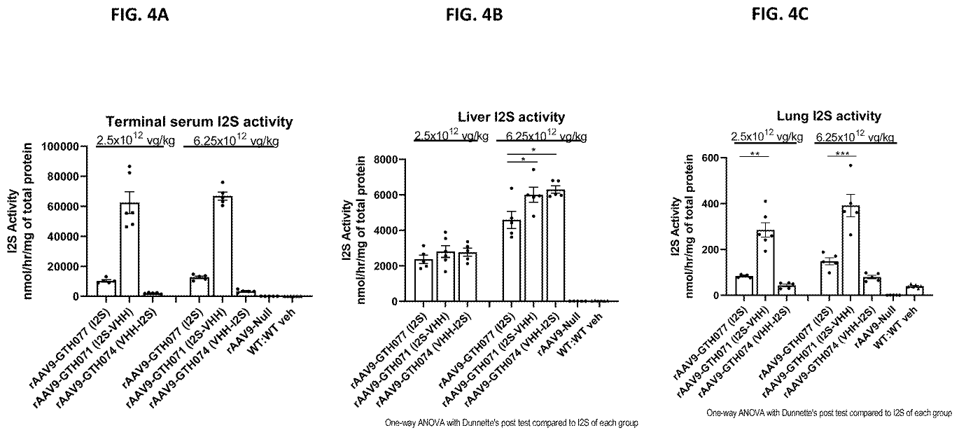

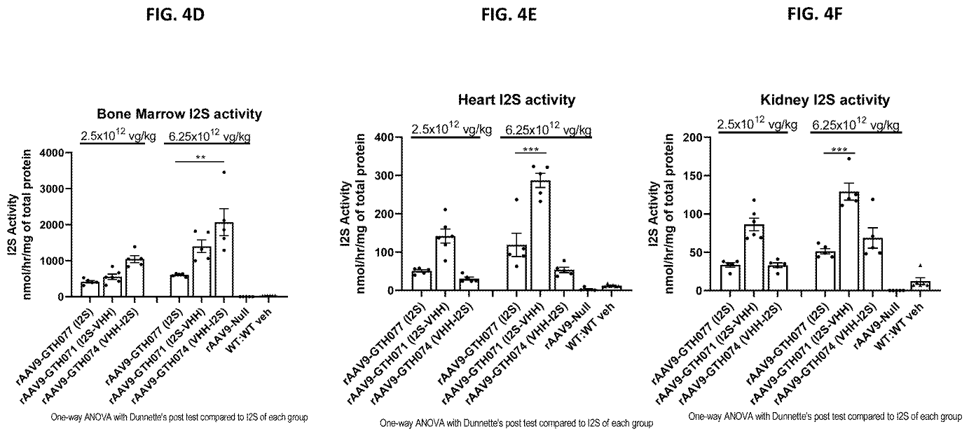

In contrast, levels of hI2S in circulation was the lowest after administration of rAAV9-GTH074 (VHH-I2S) compared to that of rAAV9-GTH077 (I2S) or rAAV9-GTH071 (I2S-VHH) at equivalent doses (FIG. 4), likely due to increased uptake by tissues. Strong hI2S activity was detected in all key tissues examined including the lungs (FIG. 4C), liver (FIG. 4B), bone marrow (FIG. 4D), heart (FIG. 4E), and kidney (FIG. 4F). hI2S activity in peripheral tissues results in the normalization of heparan sulfate levels in these tissues as well as in the spleen and quadricep muscles to WT levels (Table 7). This clearly demonstrated that the N terminally tagged VHH-I2S protein produced in liver was able to penetrate across the BBB more efficiently, was taken up by neurons, and robustly cross-corrected HS GAGs accumulation in the brain and CSF significantly better than untagged I2S, as well as resulted in substantial clearance of GAGs key peripheral organs, within 4 weeks post administration.

Gene Therapy Constructs Expressing Ubiquitous Promoter Driven VHH-I2S Show Significantly Higher Brain Activity and GAG Clearance than Constructs Expressing Untagged I2S.

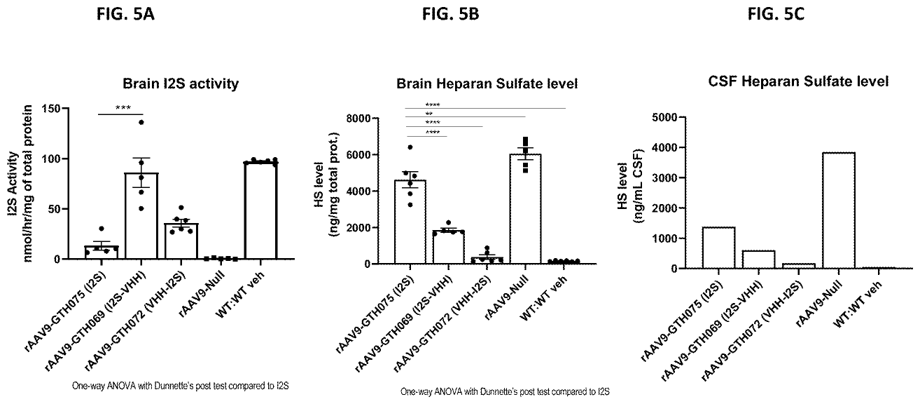

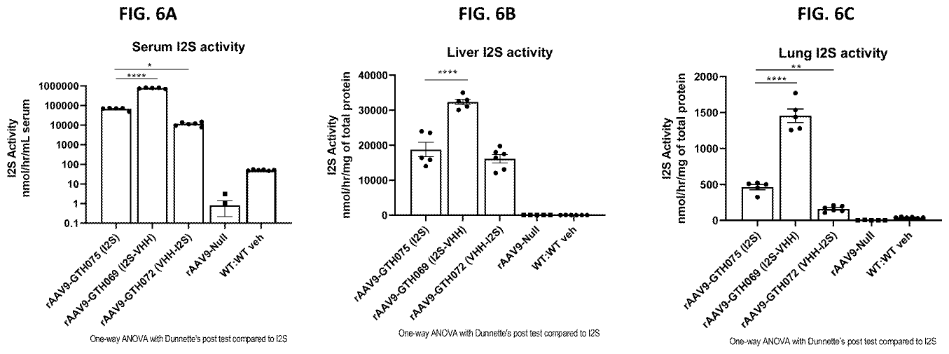

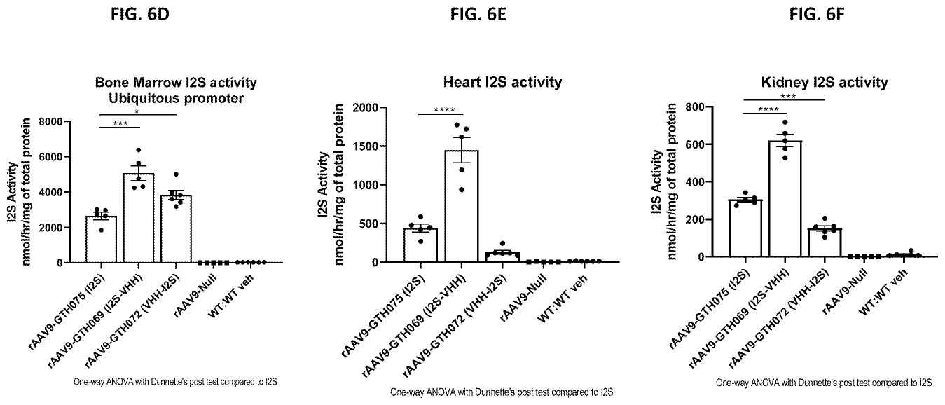

The same pattern of I2S expression and GAG reduction was observed in animals treated with rAAV9 vectors expressing I2S driven by a ubiquitous promoter at a 2.5×10¹² vg/kg dose. Significantly higher hI2S activity was observed in the brain of mice treated with rAAV9-GTH069 (I2S-VHH) or rAAV9-GTH072 (VHH-I2S) over mice treated with rAAV9-GTH075 (I2S) (FIG. 5). Consequently, significantly greater GAG reduction was also observed in the brain and CSF in animals treated with rAAV9-GTH072 (VHH-I2S) compared to other groups (FIG. 5). Strong hI2S exposure was measured in all tissues tested (FIGS. 6A-F) and resulted in normalization of GAG levels in peripheral tissues such as the liver and spleen (Table 7).

TABLE 7
Percentage HS GAGs remaining in CNS and key peripheral tissues after treatment of IdsKO mice with indicated molecules.

| | | | | | | | | Bone | Quad | |
|---|---|---|---|---|---|---|---|---|---|---|
| Dose | Tool vector molecules | Brain | CSF | Lungs | Liver | Heart | Spleen | Marrow | Muscle | Kidney |
| 2.5 × 10¹² | rAAV9-GTH077 (I2S) | 85% | 37% | 3% | 1% | 6% | 5% | 3% | 3% | 3% |
| vg/kg | rAAV9-GTH071 (I2S- | 81% | 42% | 2% | 1% | 6% | 5% | 3% | 4% | 2% |
| | VHH) | | | | | | | | | |
| | rAAV9-GTH074 (VHH- | 6% | 5% | 9% | 1% | 6% | 4% | 3% | 6% | 2% |
| | I2S) | | | | | | | | | |
| 6.25 × 10¹² | rAAV9-GTH077 (I2S) | 91% | 43% | 2% | 1% | 5% | 6% | 3% | 3% | 2% |
| vg/kg | rAAV9-GTH071 (I2S- | 64% | 38% | 2% | 1% | 4% | 5% | 3% | 2% | 1% |
| | VHH) | | | | | | | | | |
| | rAAV9-GTH074 (VHH- | 8% | 5% | 6% | 1% | 7% | 5% | 3% | 3% | 3% |
| | I2S) | | | | | | | | | |
| | rAAV9-MY011 (Null) | 100% | 100% | 100% | 100% | 100% | 100% | 100% | 100% | 100% |
| Vehicle | WT:WT | 3% | 1% | 1% | 0% | 4% | 4% | 4% | 1% | 2% |
| 2.5 × 10¹² | rAAV9-GTH075 (Cbh-I2S) | 71% | 36% | 3% | 1% | 4% | 5% | 3% | 3% | 1% |
| vg/kg | rAAV9-GTH069 (Cbh-I2S- | 31% | 16% | 3% | 1% | 4% | 4% | 3% | 4% | 2% |
| | VHH) | | | | | | | | | |
| | rAAV9-GTH072 (Cbh- | 6% | 4% | 2% | 1% | 4% | 4% | 2% | 4% | 2% |
| | VHH-I2S) | | | | | | | | | |

Sustained hI2S Activity Observed in Serum Throughout 4-Week Study in IdsKO Mice Treated with Gene Therapy Constructs Expressing I2S

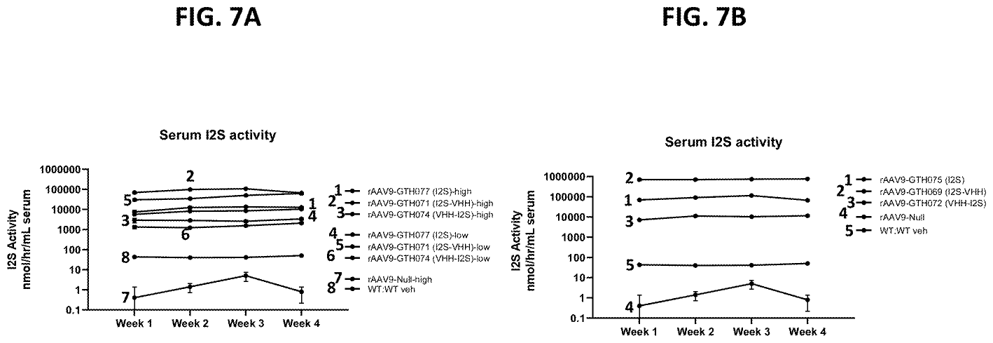

All the rAAV9 based GT constructs with either promoter resulted in steady supraphysiological levels of hI2S activity in circulation sustained throughout the study from week 1 to week 4 compared to WT normal mice (FIGS. 7A-7B). At a 2.5×10¹² vg/kg dose, gene targeting (GT) constructs with a ubiquitous promoter (GTH075, GTH069, GTH072), resulted in higher serum hI2S activity than GT constructs with a liver specific promoter (GTH077, GTH071, GTH074).

### Methods and Materials

Identification of a single-domain anti-TfR1 antibody: Various approaches using phage and yeast display techniques are used for the identification of an anti-TfR1 single-domain antibody (sdAb) utilizing a combination of in-vivo and in-vitro approaches to ensure a large and diverse binder sets from libraries.

Discovery of sdAbs can come from llama immunizations (immune libraries) or naïve synthetic library approaches.

sdAb can be derived from llama, alpaca, camelid, or can come from synthetically designed sources. They can have fully llama, humanized, or human frameworks depending on the libraries that were used. They can have camelid, human, or a combination thereof CDR sequences.

Production of recombinant enzyme-antibody fusion proteins: Plasmids were generated containing polynucleotide sequences for different his-tagged enzyme-sdAntibody (sdAb) fusion proteins (e.g. VHH-I2S and I2S-VHH). The sdAb was either fused at the N- or C-terminus of the enzyme and connected by a short linker (5G).

Plasmids were introduced into host cells (CHO cells) using a flow electroporation system, such as the MaxCyte GT®, MaxCyte VLX®, or MaxCyte STX® transfection systems. The transfected host cells expressed the enzyme-sdAb protein at a level sufficient to allow for fed-batch cell culture. Conditioned media from transfected cells was collected 6 days post transfection and used for purification.

Proteins were purified using an affinity column followed by a SEC column. 5 ml complete His-tag purification column (Cat #06781535001) was pre-equilibrated with PBS. 1 L of CM was loaded on column and chased with PBS. Column was washed with PBS, 1M NaCl followed by PBS. Protein was eluted with PBS, 250 mM imidazole pH7.5. Elution peak was collected and loaded on HiLoad® 16/600 Superdex® 200 pg (Cat #28-9893-35) pre-equilibrated with PBS. 2 ml fractions were collected along 1.2 CV. Elution peak fractions were analyzed on SDS-PAGE and pooled based on peak shape and appearance on the gel. Purified proteins were formulated in 1×PBS pH 7.4.

Surface Plasmon Resonance (SPR) assays: Binding affinity between human/mouse receptor with Iduronate 2 Sulfatase or idursulfase (I2S) tagged with an anti-transferrin receptor binding single domain antibody (VHH) was determined using the Biacore 8K+ instrument (Cytiva; Danaher Corporation). In this experiment, samples were immobilized on a series S sensor chip CM5 (Catalog #29104988) via amine coupling. Samples immobilized (ligand) were i) I2S-VHH ii) VHH-I2S iii) I2S as negative control iv) Holo transferrin peptide as positive control. 1 ug/mL of ligand, reconstituted in Acetate pH 4.0, was immobilized on individual flow cells for 420 s, yielding a final immobilization level of approximately 350 Response Units. Recombinant Human TfR Protein (R&D Systems; Catalog #2474-TR) and Mouse TfR1 (TR06671428-001; Takeda) were injected on this surface (as analyte) with a concentration range of 5, 2.5, 1.25, 0.625 & 0.156 ug/mL. Analyte series was injected for 120 s, followed by a dissociation phase for a duration that yields at least 10% dissociation of analyte. Resulting sensograms were analyzed using the 1:1 Binding Model.

Plasmids: Plasmids expressing human idursulfase (I2S) with and without an anti-hTfR1 receptor binding single domain antibody (VHH) under a liver specific promoter were tested first in Huh7 cells via transfection. Plasmids expressing human idursulfase (I2S) with and without an anti-hTfR1 receptor binding single domain antibody (VHH) under a liver specific promoter or a ubiquitous promoter were tested in a mouse model of Hunter disease.

Transfection: Huh7 (human hepatoma) cells were transfected with plasmids expressing hI2S using lipofectamine 3000 reagent kit (Thermo Fisher Scientific) as per manufacturer's instructions. Briefly, cells were seeded at 125,000 cells per well in a 12 well plate format with 1 mL per well growth media and maintained at 37° C., 5% CO₂ overnight. Next day, a fresh media was added (1 mL per well) before transfection. For each plasmid, lug of plasmid DNA was added to 2 ul of P3000 reagent, 1.5 ul of lipofectamine 3000 and enough OptiMEM media to make up 100 ul and incubated at room temperature for 10-15 mins. This mixture was then added to the cells and incubated at 37° C., 5% CO₂ overnight. Next day, media was refreshed, and cells incubated for another day before collecting the supernatant for idursulfase (I2S) activity analysis.

Viral Vectors: The rAAV9 stocks were produced using HEK-293T cells by the adenovirus-free, triple-plasmid co-transfection method and purified using column chromatography and cesium chloride ultracentrifugation. Titers of viral genome (vg) particle number were determined by droplet digital PCR. The following recombinant adeno associated viral vectors expressing I2S under either a liver specific promoter (GTH071, GTH074 and GTH077) or a ubiquitous promoter (GTH069, GTH072 and GTH075) were prepared in the formulation buffer consisting of 1.5 mM KH₂PO₄, 2.7 mM KCl, 8.1 mM Na₂HPO₄, 136.9 mM NaCl and 0.001% Pluronic F-68. The constructs GTH069 and GTH071 expressed I2S with a C terminal single domain antibody (VHH) tag that binds to the transferrin receptor (TfR1), while GTH072 and GTH074 expressed I2S with a N terminal VHH tag. Plasmids GTH075 and GTH077 expressed untagged I2S. A null vector with rAAV9 capsid (rAAV9-MY011) was used as a control.

Animals: Mus musculus, IdsKO mouse model was generated by a targeted disruption of the X-linked I2S gene locus (a replacement of exon 5 and part of exon 4 with a neomycin-resistance gene expression cassette, a 1.5 kb deletion) completely abolishing endogenous I2S enzyme activity (Muenzer et al. 2002). IdsKO mice exhibit a progressive, degenerative phenotype comprised of both peripheral and neurological symptoms that correspond to the clinical disorder. Glucosaminoglycan (GAG) accumulation, resulting from limited I2S activity, is evident in urine, liver, kidney, spleen, heart, and brain and is accompanied by vacuolization and increased lysosomal size evidenced by LAMP-1 staining in the IdsKO model by 3 months (Garcia et al. 2007; Cardone et al. 2006).

In vivo Studies: 10-12 weeks old I2Sko male mice were intravenously administered once with gene therapy constructs expressing I2S, at either 2.5×10¹² vg/kg or 6.25×10¹² vg/kg dose and monitored for 4 weeks post dose. WT:WT sibling mice with the same genetic background were used as control and were administered with vehicle only. Mice were assigned to each test article group in a semi-randomized process based on pre-dose body weights to ensure balanced groups. Serum was collected during the study at multiple time points. Blood was collected via submandibular or tail vein bleed during the study and via cardiac puncture at termination and processed to collect serum. At the end of the study, terminal serum was collected, and mice were perfused for collection of organs, including liver, lungs, bone marrow, kidney and heart. The tissues were then either snap frozen in dry ice and stored at −80° C. or fixed with 10% neutral buffered formalin (NBF) for histological evaluation. Analytical evaluations included measurement of I2S enzyme activity, and analyses of substrate levels in serum and various tissues.

Idursulfase Activity: Tissues were homogenized in lysis buffer containing 10 mM HEPES with 0.5% Triton-X 100 and 1.5× Halt protease inhibitor cocktail, EDTA free, centrifuged and supernatant collected for analytical assays. Idursulfase activity in supernatant or serum was measured using a two-step activity assay using a fluorescent substrate. During the first step (1), idursulfase (I2S), a lysosomal enzyme, hydrolyzes 4-methylumbellifery α-L-iduronide-2-sulfate (4-MUS) to 4-methylumbelliferyl α-L-iduronide (MUBI). During the second step (2), another lysosomal enzyme alpha-L-iduronidase (IDUA) hydrolyzes MUBI to the final product, 4-methylumbelliferone (4-MU). 4-MU emits fluorescence, and the signal can be quantified. Briefly, 10 ul of biological samples were incubated with 20 uL 4-MUS solution at 37° C. for 60 minutes. At the end of this 1 hour, 45 ul of IDUA solution was added and incubated for 4 hours at 37° C. The enzymatic reaction was stopped by addition of 200 uL 0.5M sodium carbonate stop solution, pH 10.7. The 4-MU product was measured at the excitation wavelength 365 nm and emission wavelength 450 nm by a fluorescence plate reader. The concentrations of 4-MU in testing samples were calculated from the 4-MU calibration curve in the same plate. Tissue activity was normalized to total protein concentration determined by BCA assay (Thermo Scientific, catalog #23225).

Heparan Sulfate Glycosaminoglycan Quantification: Substrates from serum and tissue samples were analyzed using an LC-MS method. Samples were extracted first using Chloroform:Methanol (v/v 2:1) and formic acid before running in HPLC and LC-MS/MS (Applied Biosystem API5000, Turbo Ion Spray Ionization, positive-ion mode).

Methods of Statistical Analysis: All statistical analyses were performed within GraphPad Prism software version 8, using one-way analysis of variance (ANOVA), or two-way ANOVA with multiple comparisons, including but not limited to Dunnette's and Turkey's post-test. Analysis of treated groups were compared to controls as indicated on each graph. P-value <0.05 is labeled as “*”, <0.01 “**”, <0.001 “***” and <0.0001 “****”.

### Example 2—Assessment of a Plasmid Expressing GCB-VHH in a Mouse Model of Gaucher Disease

Gaucher disease is characterized by a deficiency of the lysosomal enzyme β-glucocerebrosidase (GCB) and subsequent accumulation, predominantly in the liver, spleen, and bone marrow, of the enzyme substrate, glucocerebroside.

This example shows that administration of GCB-VHH resulted in high brain exposure of GCB in a D409V mouse model of Gaucher disease. D409V mouse model carries a homozygous single point mutation in the murine Gba1 gene and results in residual GCB activity of 5% in the periphery and 18% in the CNS, accumulation of the glycolipids, GL1 and lyso-GL1 (Sardi et al., 2013, PNAS 110(9):3537-42). Substrate accumulation and the associated neurological symptoms, including seizure, motor dysfunction and memory impairment, are progressive and significant beginning at 10-14 weeks (Dai et al, 2016, PLoS One 11(9): e0162367).

Presence of the VHH Tag does not Hamper GCB Expression, Secretion or Catalytic Activity

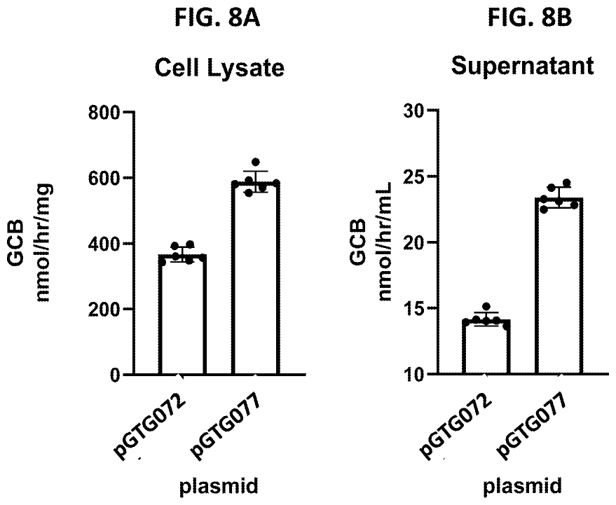

Plasmids expressing GCB with an anti-transferrin receptor binding nanobody (VHH) tag at the C terminus (pGTG077) or without any tags (pGTG072) were transfected in Huh7 (human liver) cells and then cell lysates and supernatants were collected for analyses of GCB activity. The fusion tags did not suppress GCB activity or secretion compared to untagged GCB when assessed from cell lysates (FIG. 8A) or supernatants (FIG. 8B).

Hydrodynamic tail veil injections (HTV) injection of a plasmid expressing GCB-VHH resulted in significantly higher brain exposure of GCB compared to a plasmid expressing untagged GCB in D409V mice.

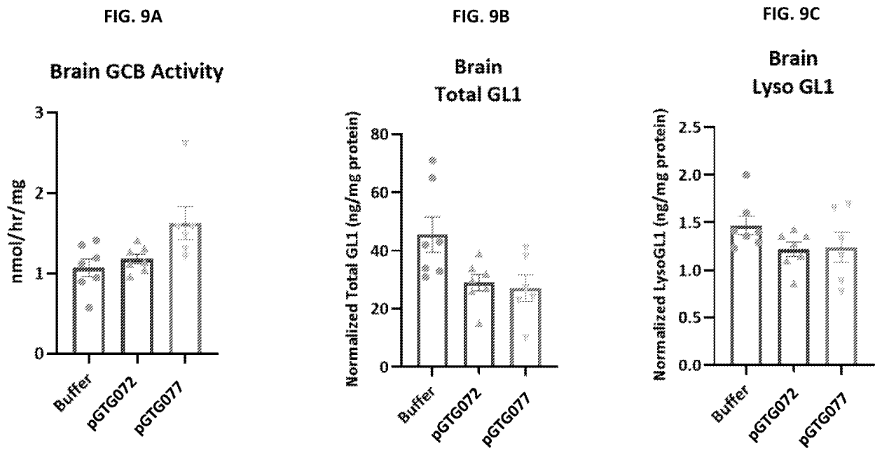

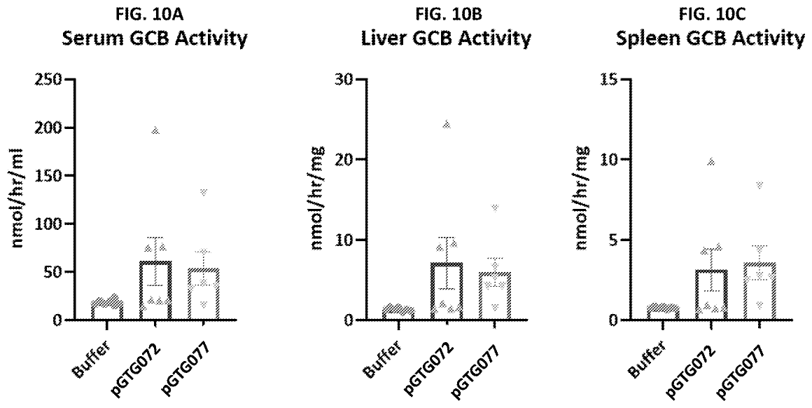

D409V mice that were injected with the plasmid pGTG077 using the hydrodynamic tail veil injections (HTV) described in the “Methods and materials” section below had significantly higher levels of GCB activity in the brain compared to mice injected with pGTG072 (FIG. 9A). This resulted in a small reduction in GL-1 (FIG. 9B) and lyso-GL1 levels (FIG. 9C) in the brain. This higher brain exposure was not due to increased GCB production or secretion since GCB levels in the liver (FIG. 10B) and in circulation (as assessed via serum; FIG. 10A) was comparable between the two groups. GCB was also found in other tissues examined such as the spleen (FIG. 10C), suggesting uptake of GCB into such tissues.

### Methods and Materials

Plasmids: Plasmids expressing human glucocerebrosidase (GCB) with and without an anti-hTfR1 receptor binding single domain antibody (VHH) tag under a liver specific promoter were tested first in Huh7 cells via transfection and then in a mouse model of Gaucher disease. pGTG072 expressed untagged GCB while pGTG077 expressed GCB-VHH.

Transfection: Huh7 (human hepatoma) cells were transfected with plasmids expressing GCB using lipofectamine 3000 reagent kit (Thermo Fisher Scientific) as per manufacturer's instructions. Briefly, cells were seeded at 125,000 cells per well in a 12 well plate format with 1 mL per well growth media and maintained at 37° C., 5% CO₂ overnight. Next day, a fresh media was added (1 mL per well) before transfection. For each plasmid, lug of plasmid DNA was added to 2 ul of P3000 reagent, 1.5 ul of lipofectamine 3000 and enough OptiMEM media to make up 100 ul and incubated at room temperature for 10-15 mins. This mixture was then added to the cells and incubated at 37° C., 5% CO₂ overnight. Next day, media was refreshed, and cells incubated for another day before collecting the supernatant for GCB activity analysis.

Animals: Mus musculus, D409V mouse model was used in this example.

Hydrodynamic gene delivery: Hydrodynamic tail veil injections (HTV) were conducted as described by Zhang et al., Hum. Gene Ther. 10:1735-37 (1999) and Herweijer and Wolff, Gene Ther. 14:99-107 (2007). Briefly, mice were administered test articles (plasmids) in a volume of solution that is equal to 8% of the animal weight on a mL per gram basis. The injection period was no more than 8 seconds.

In vivo studies: 8-12-week-old male D409V mice were administered with 50 ug plasmid DNA each via hydrodynamic gene delivery by tail vein injection. An arm was included in the study where D409V mice were injected with buffer only as a negative control. The animals were sacrificed 2 days post injection. Serum was collected by cardiac puncture at terminal endpoint and tissues such as the brain, liver and spleen were collected after perfusion with PBS. Samples were snap frozen and stored at −80° C. Serum and tissue samples were analyzed for GCB activity and GL-1 and lyso-GL1 levels.

GCB activity assay: Tissues were lysed in cold lysis buffer (10 mM HEPES; 0.5% Triton-X 100 and 2× Halt protease inhibitor cocktail, EDTA free (Thermo Fisher, Halt protease inhibitor cocktail, 100×, EDTA free, cat #78425) at a 250 mg tissue per mL concentration and then freeze-thawed thrice. Lysate was spun down at 4° C. for 10 mins at 16,000×g and supernatant collected for BCA (to normalize protein levels) and activity assays.

GCB activity was assessed using a fluorometric assay that measures its ability to hydrolyze the substrate 4-MU-glucopyranoside (4-MU-GPS, Sigma, Catalog #M3633) to 4-methylumbelliferone (4-MU). Briefly, biological samples diluted appropriately in sample buffer (0.05 M citric acid, 0.1 M sodium phosphate, 2 mg/mL BSA, pH 5.0) were incubated with 4-MU-GPS in substrate solution (0.05 M citric acid, 0.1 M sodium phosphate, 0.3% tween-20, 0.6% sodium taurocholate, pH 5.0, at 5× above Km=1 mM) at 37° C. for 60 minutes. The enzymatic reaction was stopped by the addition of glycine carbonate stop solution, pH 10.7 (333 mM Glycine, 207 mM Sodium Carbonate). The 4-MU product was measured at the excitation wavelength 360 nm and emission wavelength 465 nm by a fluorescence plate reader. The concentrations of 4-MU in testing samples were calculated from the 4-MU calibration curve in the same plate. An equivalent plate was run with lysosomal glucocerebrosidase inhibitor Conduritol B Epoxide (CBE, MW: 162.1 g/mol, Millipore, Catalog #234599) at a final concentration of 3 mM to subtract any non-lysosomal residual activity in all biological samples. One unit of activity is expressed as the conversion of 1 nmole of 4-MU-GPS to 4-MU in 1 hour at 37° C.

GL1 (glucosylceramide) and lyso-GL1 (glucosylsphingosine) quantification: Substrates from serum and tissue samples were analyzed using an LC-MS method. Samples were extracted first using Chloroform:Methanol (v/v 2:1) and formic acid before running in HPLC and LC-MS/MS (Applied Biosystem API5000, Turbo Ion Spray Ionization, positive-ion mode).

### Example 3—Mucopolysachharidosis IIIA, Sanfilippo a (San A) Disease

Mucopolysachharidosis type IIIA, also known as Sanfilippo A (San A) disease, is characterized by mutations in the SGSH gene. These mutations reduce or eliminate the function of the associated enzyme, ultimately disrupting the breakdown of heparan sulfate.

This example shows that injection of a plasmid expressing SGSH-VHH in WT mice results in high brain exposure of SGSH.

Presence of the VHH Tag does not Hamper SGSH Expression, Secretion, or Catalytic Activity

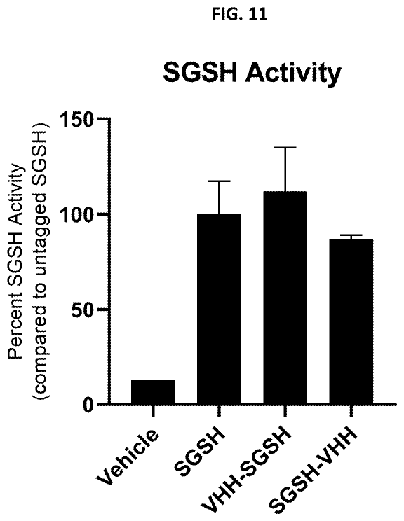

Plasmids expressing SGSH with an anti-transferrin receptor binding nanobody (VHH) tag were transfected in Huh7 (human liver) cells and then cell lysates and supernatants were collected for analyses of SGSH activity. The fusion tags did not suppress SGSH activity or secretion compared to untagged SGSH (FIG. 11).

HTV injection of a plasmid expressing SGSH-VHH resulted in significantly higher brain exposure of SGSH compared to a plasmid expressing untagged SGSH in WT mice.

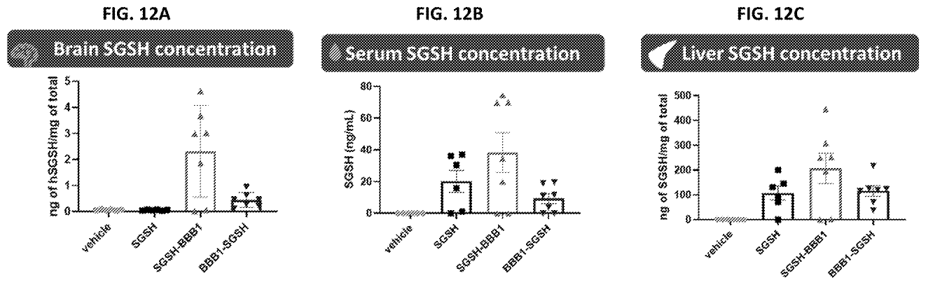

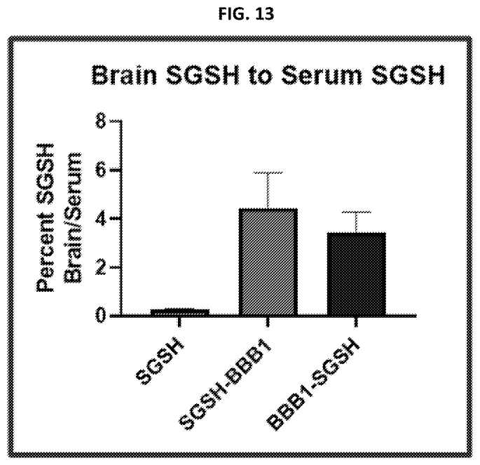

WT mice injected with the plasmid pBBB1-SGSH had significantly higher levels of SGSH activity in the brain compared to mice injected with pSGSH (FIG. 12A). Higher levels of SGSH-VHH were also found in the liver (FIG. 12C) and serum (FIG. 12B) compared to SGSH or VHH-SGSH. However, the brain exposure to serum ratio was significantly greater for SGSH-VHH and VHH-SGSH compared to VHH only (FIG. 13).

### Methods and Materials

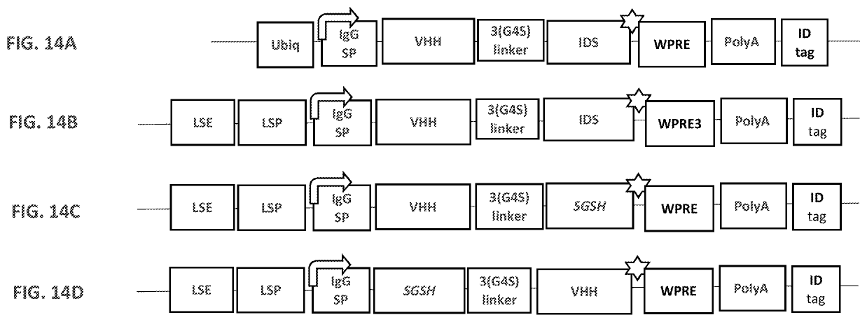

Plasmids: Plasmids expressing human sulfamidase (SGSH) with and without an anti-hTfR1 receptor binding single domain antibody (VHH) tag under a ubiquitous promoter were tested first in Huh7 cells via transfection and then in a mouse model of SanA disease. pSGSH-BBB1 expressed SGSH tagged with VHH at the C terminus while pBBB1-SGSH expressed SGSH with a N-terminal VHH tag. pSGSH expressed untagged SGSH. Example constructs are presented in FIGS. 14A-14D.

Transfection: Huh7 (human hepatoma) cells were transfected with plasmids expressing SgSh using lipofectamine 3000 reagent kit (Thermo Fisher Scientific) as per manufacturer's instructions. Briefly, cells were seeded at 125,000 cells per well in a 12 well plate format with 1 mL per well growth media and maintained at 37° C., 5% CO₂ overnight. Next day, a fresh media was added (1 mL per well) before transfection. For each plasmid, lug of plasmid DNA was added to 2 ul of P3000 reagent, 1.5 ul of lipofectamine 3000 and enough OptiMEM media to make up 100 ul and incubated at room temperature for 10-15 mins. This mixture was then added to the cells and incubated at 37° C., 5% CO₂ overnight. Next day, media was refreshed, and cells incubated for another day before collecting the supernatant for SgSh activity analysis.

Animals: Mus musculus, wild type C57B16 animals were used in this example.

Hydrodynamic gene delivery: Hydrodynamic tail veil injections (HTV) were conducted as previously described.

In vivo studies: 8-12-week-old male WT mice were administered with 50 ug plasmid DNA each via hydrodynamic gene delivery by tail vein injection. An arm was included in the study where mice were injected with buffer only as a negative control. The animals were sacrificed 2 days post injection. Serum was collected by cardiac puncture at terminal endpoint and tissues such as the brain and liver were collected after perfusion with PBS. Samples were snap frozen and stored at −80° C. Serum and tissue samples were analyzed for SgSh concentration.

SgSh ELISA Assay: Immuno-quantification of SGSH was performed using Meso Scale Discovery (MSD) technology. A total of 100 μL of anti-SGSH antibody R3074 from Shire (10 μg/mL) diluted in 0.05 M carbonate-bicarbonate buffer (pH 9.6) was used to coat multi-well plates at 4° C., 200 rpm, overnight. Following an incubation with MSD-A blocking buffer for 1 h, samples or standards were incubated at 37° C. for 1 h and then Sulfo tag-conjugated 100 μL rabbit anti-SGSH antibody R3074 (10 μg/mL) at 37° C. for 1 h. The plate was then incubated with 2× read buffer and read using an MSD instrument. Wash step in between incubations was performed using 0.05% PBS-Tween.

SgSh Activity Assay: SGSH activity was measured by combining 10 μl of sample (cell/tissue lysate) with 20 μl of 1 mM 4MUGlcNS in Michaelis' barbital CH₃COONa buffer (29 mM sodium barbital, 29 mM CH₃COONa, and 0.68% (w/v) NaCl; pH 6.5) and incubated at 37° C. for 17 h before addition of 16 μl of PiCi buffer (400 mL of milliQ H2O, 10.36 g of Sodium Phosphate Dibasic, 2.3 g Citric Acid, pH to 6.5) with a-glucosidase 500 U/mL in H2O). Samples were incubated for a further 5 h at 37° C. before the reaction was stopped with the addition of 0.2 mL of glycine buffer (0.5M Na₂CO₃/NaHCO₃ 0.025% Triton X-100; pH 10.7). The samples were aliquoted into black microtiter plates and fluorescence determined using a Spectramax M3 reader with an excitation wavelength of 360 nm and an emission wavelength of 460 nm. Enzyme activity was determined by relating the fluorescence of the sample to that of a known concentration of 4 MU and expressed in nmol/mg protein.

### Example 4—In Vitro Model for Blood Brain Barrier Transcytosis

In vitro models were used to test the transcytosis ability of SGSH-BBB1 (SGSH tagged with VHH at the C terminus) and BBB1-SGSH (SGSH with a N-terminal VHH tag), described in Example 3, above, as compared to a control SGSH construct that does not comprise a VHH. First, the Mimetas 3D cell culture organ-on-a-chip technology was used to measure transcytosis of SGSH, SGSH-BBB1, and BBB1-SGSH. FIGS. 15A-15B show the percentage of protein passed through the chamber after day 7 of treatment with 62.7 μg/ml (FIG. 15A) or 31.25 μg (FIG. 15B) of SGSH-BBB1 or BBB1-SGSH fused protein. Although there is some non-fused protein measured from transcytosed media, it is clear that the fused protein showed almost a 2- to 3-fold increase in abundance in the lower chamber, indicating that these constructs are capable of improved transcytosis, relative to control.

Next, a transwell model was used to measure transcytosis. Transwell plates were prepared with a total of six, twelve, or twenty-four wells containing a porous filter insert, which divides the transwell into upper apical or blood compartment and lower basolateral or brain compartment. A uniform layer of brain endothelial cells (BECs) were seeded on the top of the filter membrane and coated with an extracellular matrix (ECM)-derived protein collagen I and with or without seeding with astrocytes in the apical or basolateral sides. hBMEC cells were seeded in the upper chamber.

FIG. 16C shows a representative image of TEER value after 3 days post seeding with hBMEC cells compared to cell free wells. SGSH activity measured from transfected media using both fused and non-fused SGSH plasmid constructs showed similar level of activity (FIG. 16B). The percentage of protein that passed through the chamber post 3 days of treatment using transfected media collected from Huh7 cells was found to be over 3-fold higher for the BBB1-SGSH construct as compared to the SGSH-BB1 construct and the control (FIG. 16C), and a similar increase was observed when purified BBB1-SGSH was used (FIG. 16D). Transcytosis using this model was dose dependent (FIG. 16E).

Taken together, these results illustrate that secreted protein following transfection of human liver cells closely mimics the BBB transcytosis observed with purified protein. Further, N-terminal fused protein showed better efficacy in terms of higher transcytosis.

### Example 5—Characterization of MPSIIIA Patient Fibroblasts

Fibroblasts obtained from mucopolysachharidosis type IIIA (MPSIIIA) patients were characterized for glycosaminoglycans (GAG) accumulation. Mass spectrometry was used to detected GAG following two days in culture. GAG was observed to accumulate at a higher level in patient fibroblasts (P1, P2, and P3) as compared to normal cells (C1 and C2) (FIG. 17A). Time and dose dependent accumulation of SGSH was observed in fibroblasts (FIGS. 17B-17C). With increased days of culture an increase in SGSH activity in both healthy cells and patient's fibroblasts was observed.

Next, the abundance of lysosomes in MPSIIA-patient derived fibroblasts was compared to healthy fibroblast controls. Cells were seeded at 75 k/well in 12-well glass bottom plates and cultured for five days. Cells were then stained with lysotracker red and Hoechst. Representative images show increased abundance of lysosomes in patient fibroblasts (FIGS. 18E-18J) as compared to cells from healthy subjects (FIGS. 18A-18D).

### Example 6—In Vivo Mouse Model for Blood Brain Barrier Transcytosis

A proof-of-concept study was designed in wild-type mice to assess the pharmacokinetics and BBB penetrance of a TfRc binding tag fused to SGSH protein. Transfection of the liver was achieved via the hepatic vein by pressure from injection of a large bolus of plasmid into the tail vein. Brain, liver, serum, spleen, and kidney samples were collected at day 2 post injection. Human SGSH protein levels and activity were assayed and compared to untreated controls.

Two days after injection, hydrodynamic tail vein injection successfully delivered the GGT plasmids to the liver of WT type animals resulting in the expression of SGSH with and without a BBB1 fusion tag (FIG. 19A). The SGSH was secreted into circulation, providing systemic delivery of SGSH protein (FIG. 19B). Antibodies used for this measurement were specific to human SGSH, resulting in little or no background levels in the control mice. SGSH fused with BBB1 was quantified in the brain by MSD (FIG. 19C). The SGSH activity levels in the brain were found to be elevated above wild-type levels when the animals were treated with SGSH-BBB1 fusion constructs, with an increase in brain activity by at least 30% of wild-type activity (130% total) is achieved by the 2-day HTV (FIG. 19D). As expected, some activity is observed in control mice, as the assay detects both human and mouse SGSH activity, and the control mice are healthy mice that are expected to have normal SGSH activity. Immuno-histochemistry for SGSH shows the brain biodistribution in the cortex (FIGS. 20A, 20C, and 20E) and the hippocampus (FIGS. 20B, 20D, and 20F) of SGSH C- and N-terminal fusion constructs.

Fluorescent immuno-histochemistry was used to determine the biodistribution of SGSH in the brain two days after HTV administration. Throughout the sections, SGSH colocalized with GFAP staining, suggesting strong biodistribution to astrocytes, a key target cell type of MPSIIIA (data not shown). Some neuronal colocalization of SGSH was also observed.

To further test the effects of the SGSH BBB1 constructs, a MPSIIIA mouse model was developed that comprises a knock-in point mutation that leaves animals with about 3-4% of wild type levels of SGSH activity in homozygotes (FIGS. 21A-21D). The model was brought out of cyro-preservation and validated for reduced SGSH activity and lysosomal dysfunction by LAMP1 IHC a lysosomal marker (FIGS. 21E-21G). The mouse model can be used to further measure substrate reduction by the various plasmids described herein.

What is claimed is:

1. A gene therapy vector comprising:
(a) a 5′ inverted terminal repeat (ITR)
(b) a promoter,
(c) a transgene comprising (i) a nucleotide sequence encoding a biologically active polypeptide and (ii) a nucleotide sequence encoding a TAG, and
(d) a 3′ ITR.

2. The gene therapy vector of claim 1, wherein the biologically active polypeptide comprises a therapeutic enzyme.

3. The gene therapy vector of claim 1 or 2, wherein the biologically active polypeptide reduces the levels of a substrate in the central nervous system.

4. The gene therapy vector of any one of claims 1 to 3, wherein the biologically active polypeptide has an idursulfase activity, has a glucocerebrosidase activity or has a sulfamidase activity.

5. The gene therapy vector of any one of claims 1 to 4, wherein the biologically active polypeptide is an idursulfase (IDS), a glucocerebrosidase (GCB) or a sulfamidase (SGSH).

6. The gene therapy vector of any one of claims 1 to 5, wherein the TAG increases the translocation of the biologically active polypeptide across the blood brain barrier.

7. The gene therapy vector of any one of claims 1 to 6, wherein the TAG comprises an antigen-binding molecule.

8. The gene therapy vector of any one of claims 1 to 7, wherein the TAG comprises an scFv, a VHH, a vNAR, a diabody, a nanobody, a camelid antibody, or a combination thereof.

9. The gene therapy vector of any one of claims 1 to 8, wherein the TAG comprises a VHH.

10. The gene therapy vector of any one of claims 1 to 9, wherein the TAG comprises an antigen-binding molecule that specifically binds transferrin receptor 1 (TfR1).

11. The gene therapy vector of any one of claims 1 to 10, wherein the TAG comprises a VHH that specifically binds TfR1.

12. The gene therapy vector of any one of claims 1 to 11, wherein the TAG comprises a variable heavy (VH) domain comprising a VH complementarity determining region (CDR) 1, a VH-CDR2, and a VH-CDR3.

13. The gene therapy vector of claim 12, wherein the VH-CDR1 is encoded by the nucleic acid sequence set forth in SEQ ID NO: 11.

14. The gene therapy vector of claim 12 or 13, wherein the VH-CDR2 is encoded by the nucleic acid sequence set forth in SEQ ID NO: 12.

15. The gene therapy vector of any one of claims 12 to 14, wherein the VH-CDR3 is encoded by the nucleic acid sequence set forth in SEQ ID NO: 13.

16. The gene therapy vector of any one of claims 12 to 15, wherein the nucleotide sequence encoding the TAG comprises a nucleic acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% identity to SEQ ID NO: 14, 15, 16, 17, 18, 19, 20, or 21.

17. The gene therapy vector of claim 16, wherein the nucleotide sequence encoding the TAG comprises SEQ ID NO: 14, 15, 16, 17, 18, 19, 20, or 21.

18. The gene therapy vector of claim 16 or 17, wherein the nucleotide sequence encoding the TAG comprises SEQ ID NO: 14.

19. The gene therapy vector of any one of claims 1 to 18, wherein the nucleotide sequence encoding the TAG comprises SEQ ID NO: 15.

20. The gene therapy vector of any one of claims 1 to 19, wherein the nucleotide sequence encoding the biologically active polypeptide is 3′ of the nucleotide sequence encoding the TAG.

21. The gene therapy vector of any one of claims 1 to 19, wherein the nucleotide sequence encoding the biologically active polypeptide is 5′ of the nucleotide sequence encoding the TAG.

22. The gene therapy vector of any one of claims 1 to 21, wherein the (i) nucleotide sequence encoding the biologically active polypeptide is linked to the (ii) nucleotide sequence encoding the TAG further by (iii) a nucleotide sequence encoding a peptide linker.

23. The gene therapy vector of claim 22, wherein the linker is a flexible linker, a cleavable linker, a processable linker, or any combination thereof.

24. The gene therapy vector of any one of claims 1 to 23, wherein the promoter is a ubiquitous promoter.

25. The gene therapy vector of claim 24, wherein the ubiquitous promoter comprises a chicken β actin (CBA) promoter, an EF-1α promoter, a PGK promoter, a UBC promoter, an LSE beta-glucuronidase (GUSB) promoter, or a ubiquitous chromatin opening element (UCOE) promoter.

26. The gene therapy vector of claim 24 or 25, wherein the ubiquitous promoter comprises a cyto-megalo-virus (CMV) enhancer, a chicken β actin promoter (CBA), and a rabbit beta globin intron.

27. The gene therapy vector of any one of claims 1 to 26, wherein the promoter is a tissue specific promoter.

28. The gene therapy vector of claim 27, wherein the promoter is a liver specific promoter.

29. The gene therapy vector of claim 27 or 28, wherein the promoter comprises an hTTR, PGK, chicken β actin (CBA) promoter, CAG promoter, EF-1α promoter, UBC promoter, LSE beta-glucuronidase (GUSB) promoter, or ubiquitous chromatin opening element (UCOE) promoter, or any combination thereof.

30. The gene therapy vector of any one of claims 1 to 29, which is a recombinant AAV (rAAV).

31. The gene therapy vector of claim 30, wherein the rAAV comprises an AAV capsid.

32. The gene therapy vector of claim 31, wherein the AAV capsid is a wide-tropism AAV capsid.

33. The gene therapy vector of claim 31 or 32, wherein the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, AAV9 capsid, and a variant thereof.

34. The gene therapy vector of any one of claims 31 to 33, wherein the AAV capsid is AAV9.

35. The gene therapy vector of any one of claims 1 to 34, further comprising a polyA sequence, which is located 3′ of the transgene.

36. The gene therapy vector of claim 35, wherein the poly A is bovine growth hormone (BGH) polyA or a synthetic polyA.

37. The gene therapy vector of claim 36, wherein the synthetic poly A is designed in silico.

38. The gene therapy vector of any one of claims 1 to 37, further comprising a posttranscriptional regulatory element.

39. The gene therapy vector of claim 38, wherein the posttranscriptional regulatory element is located 3′ of the transgene.

40. The gene therapy vector of claim 38 or 39, wherein the posttranscriptional regulatory element is located 5′ of the polyA sequence.

41. The gene therapy vector of any one of claims 38 to 40, wherein the posttranscriptional regulatory element comprises a woodchuck hepatitis virus posttranscriptional regulatory element (WPRE).

42. The gene therapy vector of claim 41, wherein the WPRE sequence is modified.

43. The gene therapy vector of claim 41 or 42, wherein the WPRE sequence is WPRE mut6delATG.

44. The gene therapy vector of any one of claims 1 to 43, wherein the promoter comprises a shortened EF-1α promoter and one or more introns.

45. The gene therapy vector of claim 44, wherein the one or more introns are from CBA and/or rabbit β-globin genes.

46. The gene therapy vector of any one of claims 1 to 45, wherein the transgene is codon optimized.

47. A method of treating a disease or condition in a subject in need thereof, comprising administering to the subject the gene therapy vector of any one of claims 1 to 46.

48. The method of claim 47, wherein the disease or condition comprises a neuronal disease.

49. The method of claim 47 or 48, wherein the disease or condition comprises a mucopolyscahharidoses.

50. The method of any one of claims 47 to 49, wherein the disease or condition comprises Hurler syndrome (MPS I), Hunter syndrome (MPS II), Sanfilippo syndrome (MPS III), Sly syndrome (MPS VII), Gaucher disease, Metachromatic leukodystrophy, Krabbe disorder, and GM1 gangliosidosis.

51. A method of delivering a biologically active polypeptide across the blood brain barrier in a subject, comprising administering to the subject the gene therapy vector of any one of claims 1 to 46.

52. A method of reducing the level of a substrate in the central nervous system of a subject, comprising administering to the subject the gene therapy vector of any one of claims 1 to 46.

53. A method of substrate reduction in the central nervous system of a subject, comprising administering to the subject the gene therapy vector of any one of claims 1 to 46.

54. A method of treating a mucopolyscahharidoses in a subject in need thereof, comprising administering to the subject the gene therapy vector of any one of claims 1 to 46.

55. A method of treating Hurler syndrome (MPS I) in a subject in need thereof, comprising administering to the subject the gene therapy vector of any one of claims 1 to 46.

56. A method of treating Hunter syndrome (MPS II) in a subject in need thereof, comprising administering to the subject the gene therapy vector of any one of claims 1 to 46.

57. A method of treating Sanfilippo syndrome (MPS III) in a subject in need thereof, comprising administering to the subject the gene therapy vector of any one of claims 1 to 46.

58. A method of treating Sly syndrome (MPS VII) in a subject in need thereof, comprising administering to the subject the gene therapy vector of any one of claims 1 to 46.

59. A method of treating Gaucher disease in a subject in need thereof, comprising administering to the subject the gene therapy vector of any one of claims 1 to 46.

60. A method of treating Metachromatic leukodystrophy in a subject in need thereof, comprising administering to the subject the gene therapy vector of any one of claims 1 to 46.

61. A method of treating Krabbe disorder in a subject in need thereof, comprising administering to the subject the gene therapy vector of any one of claims 1 to 46.

62. A method of treating GM1 gangliosidosis in a subject in need thereof, comprising administering to the subject the gene therapy vector of any one of claims 1 to 46.

63. The method of any one of claims 47 to 62, wherein a recombinant polypeptide is expressed from the transgene outside the central nervous system, and wherein the TAG facilitates translocation of the recombinant polypeptide across the blood brain barrier into the central nervous system.

64. The method of any one of claims 47 to 63, wherein the recombinant polypeptide reduces the level of a substrate in the central nervous system of the subject.

65. The method of any one of claims 47 to 64, wherein the recombinant polypeptide reduces the level of a substrate systemically.

66. A method of treating Hunter syndrome in a subject in need thereof, comprising administering to the subject the gene therapy vector of any one of claims 1 to 46, wherein the biologically active polypeptide comprises IDS.

67. A method of treating Gaucher disease in a subject in need thereof, comprising administering to the subject the gene therapy vector of any one of claims 1 to 46, wherein the biologically active polypeptide comprises glucocerebrosidase (GCB).

68. A method of treating Sanfilippo syndrome in a subject in need thereof, comprising administering to the subject the gene therapy vector of any one of claims 1 to 46, wherein the biologically active polypeptide comprises N-Sulfoglucosamine Sulfohydrolase.


SEQUENCE LISTING (26 sequences)

SEQ ID NO: 1 - protein, 550 aa, synthetic construct
  source 1..550: note = IDS Amino Acid Sequence
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - DNA, 1653 bp, synthetic construct
  source 1..1653: mol_type = other DNA; note = IDS Nucleic Acid Coding Sequence
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 3 - DNA, 1653 bp, synthetic construct
  source 1..1653: mol_type = other DNA; note = Codon-Optimized I2S Nucleic Acid Sequence-1
atgccacccccccggaccgggagaggcctcttgtggttgggcctggtgctgagcagcgtgtgcgtggccctgggcagtgagacccaggctaactctacaacagatgccttgaatgtgctgctgatcattgtggatgacctgaggccaagtctgggctgctatggggacaaattggtgaggtcccccaacatcgaccagttggcctcccactctctcctattccaaaatgctttcgcccagcaggcagtttgtgccccctctagggtgagcttcctcactggcaggcgccctgacaccactagactgtatgactttaacagctattggagggtgcacgcaggaaacttctccacaatccctcaatacttcaaggagaatggttatgtgacaatgtctgtgggcaaggtgttccaccctggcatcagcagcaaccacaccgatgactcaccctatagttggtcttttcccccctaccatccttcatctgagaaatatgaaaacacaaaaacctgccgaggcccagacggggaactgcatgccaacctactctgtcctgttgatgtactggacgtgcccgagggcaccctccctgataagcagtccacagaacaggccattcagctgcttgaaaagatgaagacctccgcatcccccttcttcttggctgtcggctaccacaagccccatatcccctttagataccccaaggaattccagaaactgtacccactggagaacatcacacttgctcctgaccctgaagtgcctgacggactgcctccagtggcctataacccttggatggacatccggcagcgcgaggatgtgcaggctctgaacattagtgtgccttatgggcccatccctgtggactttcagaggaagattcgccagtcctactttgcctctgtatcctacctggacacacaggtgggacgcctgctgtctgcccttgatgatctgcaactggccaacagcaccattatagctttcacatcagaccatgggtgggctcttggggagcatggtgaatgggctaagtactccaacttcgatgtggcaacccatgtccctctgatcttctatgtgccaggaaggaccgcctctctgccagaggcaggtgagaagctgttcccctatctggacccttttgactccgccagccagctgatggagcctggccgacagtctatggacctggttgagctggtcagcctgtttcccacactcgctggactggctggcctgcaagtacccccacgctgcccagtgccctccttccatgtggagctttgcagggaggggaagaacctcctcaagcacttcaggttcagggacctagaggaggatccttatctgcctggaaaccccagagagcttattgcttactcccagtatccaaggcctagtgacattccccaatggaactcagacaaaccaagcctgaaagacatcaagatcatgggatactctatcaggaccattgactacaggtacactgtgtgggttggcttcaacccggatgagttcctggctaatttctctgacatacatgctggcgagctgtacttcgtggacagtgaccccctgcaggatcacaacatgtacaatgattcccaggggggtgacctcttccagcttctgatgccctaa

SEQ ID NO: 4 - DNA, 1653 bp, synthetic construct
  source 1..1653: mol_type = other DNA; note = Codon-Optimized I2S Nucleic Acid Sequence-2
atgccaccacctaggacaggcaggggcctgctttggcttggactggtgctgagctctgtctgtgttgccctgggctccgagacccaagccaactctacaaccgatgctctcaatgttctgctcatcatagtggatgacctgcggccctctctaggctgctatggagacaagttggtgcggagccccaacatagaccagctagcctctcactccctgctgttccagaatgccttcgcccagcaagctgtgtgcgccccctctagagtgtctttcctgaccgggagaaggcctgatacaacaaggctgtatgactttaacagctactggagggtgcacgcaggcaacttctccactatcccccaatacttcaaggagaatggctatgtgaccatgagcgtgggcaaggtcttccaccctggaatctcctccaaccacactgatgatagtccctactcttggtcttttcctccctatcaccctagcagtgagaagtatgagaacaccaaaacctgcagaggccctgatggggagctgcatgctaacctcctgtgtcctgtagatgtgctggacgtcccagagggcaccttgccagataagcagtctactgagcaggctatccagctgcttgagaaaatgaagacttctgcatctcccttctttctggctgttggctaccacaagcctcacatccccttcaggtaccctaaggagttccaaaagctctatcctctggaaaacatcacacttgcccccgatcctgaggtccctgacggcctcccaccagtagcctacaatccttggatggacattaggcagagagaggatgtccaggctctgaatatttctgtgccctatgggcccatcccggtggacttccagcgcaaaatcagacagtcctactttgcctctgtgagctatctggacacccaggttgggaggctcctctccgcccttgacgacctccagttggccaacagcaccattatagccttcacctctgaccacggctgggcactgggggaacacggggagtgggctaagtactctaactttgatgtggccacccacgtgcccctcatcttttatgtgcctggcaggactgccagcctgcccgaagctggggaaaaactgtttccatacctggacccttttgacagtgcttctcagctcatggaacctggccgtcagagcatggatctggtggagctagtgtccctcttcccaaccttggctggccttgctggtctccaggtgcctcctagatgcccagtcccctccttccatgttgaactctgccgtgaggggaagaatctgctgaagcacttcagattcagagacttggaggaggacccctaccttcctgggaaccccagggagttgattgcatactcccagtatcccaggccaagtgacattccccagtggaactccgacaaaccaagtctgaaggacatcaagatcatggggtacagcatcaggaccattgactacagatacacagtgtgggttggatttaacccagatgagttcttggcaaacttttctgacatccatgcaagtcagttgtattttgtggacagcgaccctctgcaggatcacaacatgtacaatgacagccagggtggggacctctttcaactcctcatgccatag

SEQ ID NO: 5 - DNA, 24 bp, synthetic construct
  source 1..24: mol_type = other DNA; note = VHH-CDR1
ggaacggacttcagtatcaatttt

SEQ ID NO: 6 - DNA, 21 bp, synthetic construct
  source 1..21: mol_type = other DNA; note = VHH-CDR2
tttactgcgactggtaacaca

SEQ ID NO: 7 - DNA, 15 bp, synthetic construct
  source 1..15: mol_type = other DNA; note = VHH-CDR3
tatatgttggacaag

SEQ ID NO: 8 - DNA, 333 bp, synthetic construct
  source 1..333: mol_type = other DNA; note = VHH-1
gaggtgcagctggtggagtctgggggaggcgtggtgcagcctggggggtctctaaaactctcctgcgtagcctcgggaacggacttcagtatcaattttatacgctggtaccgccaggctccagggaagcagcgcgagttcgtcgcaggatttactgcgactggtaacacaaactatgcagactccatgaaggggcgattcaccatctccagagacaacaccaagaacgcggtgtatctgcaaatagacagcctgaaacctgaggacacggccgtgtattactgctatatgttggacaagtggggccaggggacccaggtcaccgtctcctca

SEQ ID NO: 9 - DNA, 333 bp, synthetic construct
  source 1..333: mol_type = other DNA; note = VHH-2
gaggtgcagctggtggagtctgggggaggcgtggtgcagcctggggggtctctaaaactctcctgcgtagcctcgggaacggacttcagtatcaattttatacgctggtaccgccaggctccagggaagcagcgcgagttcgtcgcaggatttactgcgactggtaacacaaactatgcagactccatgaaggggcgattcaccatctccagagacaacaccaagaacgcggtgtatctgcaaatagacagcctgaaacctgaggacacggccgtgtattactgctatatgattgacaagtggggccaggggacccaggtcaccgtctcctca

SEQ ID NO: 10 - DNA, 333 bp, synthetic construct
  source 1..333: mol_type = other DNA; note = VHH-3
gaggtgcagctggtggagtctgggggaggcgtggtgcagcctggggggtctctacgtctctcctgcgcagcctcgggaacggacttcagtatcaattttatgagctgggttcgccaggctccagggaagggtctggagtgggtcgcaggatttactgcgactggtaacacaaactatgcagactccgttaaggggcgattcaccatctccagagacaacgcaaagaacaccctgtatctgcaaatgaatagcctgcgtcctgaggacacggccgtgtattactgctatatgttggacaagtggggccaggggacccaggtcaccgtctcctca

SEQ ID NO: 11 - DNA, 333 bp, synthetic construct
  source 1..333: mol_type = other DNA; note = VHH-4
gaggtgcagctggtggagtctgggggaggcgtggtgcagcctggggggtctctacgtctctcctgcgcagcctcgggaacggacttcagtatcaattttatacgctgggttcgccaggctccagggaagcagcgcgagttcgtcgcaggatttactgcgactggtaacacaaactatgcagactccgttaaggggcgattcaccatctccagagacaacgcaaagaacaccctgtatctgcaaatgaatagcctgcgtcctgaggacacggccgtgtattactgctatatgttggacaagtggggccaggggacccaggtcaccgtctcctca

SEQ ID NO: 12 - DNA, 333 bp, synthetic construct
  source 1..333: mol_type = other DNA; note = VHH-5
gaggtgcagctggtggagtctgggggaggcgtggtgcagcctggggggtctctacgtctctcctgcgcagcctcgggaacggacttcagtatcaattttatgcattgggttcgccaggctccagggaagggtctggagtgggtcgcaggatttactgcgactggtaacacaaactatgcagactccgttaaggggcgattcaccatctccagagacaacgcaaagaacaccctgtatctgcaaatgaatagcctgcgtcctgaggacacggccgtgtattactgctatatgttggacaagtggggccaggggacccaggtcaccgtctcctca

SEQ ID NO: 13 - DNA, 333 bp, synthetic construct
  source 1..333: mol_type = other DNA; note = VHH-6
gaggtgcagctggtggagtctgggggaggcgtggtgcagcctggggggtctctacgtctctcctgcgcagcctcgggaacggacttcagtatcaattttatgagctgggttcgccaggctccagggaagcagcgcgagttcgtcgcaggatttactgcgactggtaacacaaactatgcagactccgttaaggggcgattcaccatctccagagacaacgcaaagaacaccctgtatctgcaaatgaatagcctgcgtcctgaggacacggccgtgtattactgctatatgttggacaagtggggccaggggacccaggtcaccgtctcctca

SEQ ID NO: 14 - DNA, 333 bp, synthetic construct
  source 1..333: mol_type = other DNA; note = VHH-7
gaggtgcagctggtggagtctgggggaggcgtggtgcagcctggggggtctctacgtctctcctgcgcagcctcgggaacggacttcagtatcaattttatacgctgggttcgccaggctccagggaagggtctggagtgggtcgcaggatttactgcgactggtaacacaaactatgcagactccgttaaggggcgattcaccatctccagagacaacgcaaagaacaccctgtatctgcaaatgaatagcctgcgtcctgaggacacggccgtgtattactgctatatgttggacaagtggggccaggggacccaggtcaccgtctcctca

SEQ ID NO: 15 - DNA, 333 bp, synthetic construct
  source 1..333: mol_type = other DNA; note = VHH-8
gaggtgcagctggtggagtctgggggaggcgtggtgcagcctggggggtctctacgtctctcctgcgcagcctcgggaacggacttcagtatcaattttatacattgggttcgccaggctccagggaagggtctggagtgggtcgcaggatttactgcgactggtaacacaaactatgcagactccgttaaggggcgattcaccatctccagagacaacgcaaagaacaccctgtatctgcaaatgaatagcctgcgtcctgaggacacggccgtgtattactgctatatgttggacaagtggggccaggggacccaggtcaccgtctcctca

SEQ ID NO: 16 - protein, 536 aa, synthetic construct
  source 1..536: note = GCB (hGBA1) Amino Acid Sequence
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 17 - DNA, 1580 bp, synthetic construct
  source 1..1580: mol_type = other DNA; note = GCB (hGBA1) Coding Sequence
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgttgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactc

SEQ ID NO: 18 - protein, 502 aa, synthetic construct
  source 1..502: note = SGSH (hSGSH) Amino Acid Sequence
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 19 - DNA, 1509 bp, synthetic construct
  source 1..1509: mol_type = other DNA; note = SGSH (hSGSH) Coding Sequence
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 20 - DNA, 130 bp, synthetic construct
  source 1..130: mol_type = other DNA; note = rAAV2 5'ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 218 bp, synthetic construct
  source 1..218: mol_type = other DNA; note = 3x CRM8
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 22 - DNA, 233 bp, synthetic construct
  source 1..233: mol_type = other DNA; note = hTTR
aaatgacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcagg

SEQ ID NO: 23 - DNA, 77 bp, synthetic construct
  source 1..77: mol_type = other DNA; note = MVM intron
ctaaggtaagttggcgccgtttaagggatggttggttggtggggtattaatgtttaattaccttttttacaggcctg

SEQ ID NO: 24 - DNA, 168 bp, synthetic construct
  source 1..168: mol_type = other DNA; note = sv40PolyA
agatcttttaaaaaacctcccacacaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 25 - DNA, 133 bp, synthetic construct
  source 1..133: mol_type = other DNA; note = rAAV2 3'ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagaga

SEQ ID NO: 26 - DNA, 45 bp, synthetic construct
  source 1..45: mol_type = other DNA; note = 3XG4S Linker
ggcggtggaggcagtggtggcgggggctccgggggtggcggttca